(19) United States
(12) Patent Application Publication
(10) Pub. No.: US 2026/0183107 A1
(43) Pub. Date: Jul. 2, 2026

(54) ALIGNMENT DEVICE FOR A PROSTHETIC IMPLANT

(71) Applicant: Edwards Lifesciences Corporation, Irvine, CA (US)

(72) Inventors: Brad Buzea, Trabuco Canyon, CA (US); Brendan Michael Dalbow, Oceanside, CA (US); Camille Lazaro Villadolid, Aliso Viejo, CA (US); Izaak Rosen, Irvine, CA (US); Gonzalo German Angelico, Irvine, CA (US); William A. Maywald, Fountain Valley, CA (US); Mark Andrew Williams, Newport Beach, CA (US)

(21) Appl. No.: 19/539,573

(22) Filed: Feb. 13, 2026

Related U.S. Application Data
(63) Continuation of application No. PCT/US2024/042418, filed on Aug. 15, 2024.
(60) Provisional application No. 63/533,555, filed on Aug. 18, 2023.

(51) Int. Cl. A61F 2/24

(52) U.S. Cl. CPC A61F 2/2436

(57) ABSTRACT

An alignment device for crimping a prosthetic valve onto a valve mounting portion of a delivery apparatus is disclosed. The alignment device can include a coupling portion comprising a coupling member configured to couple with a crimping device, a support portion that tapers from a wider end that is disposed adjacent a first end of the coupling portion to a narrower end, where the support portion comprises an outward facing support surface that is configured to receive the prosthetic valve thereon, and one or more axially extending alignment members. Each alignment member extends axially outward from the first end of the coupling portion, over the support portion and to the support surface, each alignment member comprising a free end configured to abut an end of the prosthetic valve and be axially aligned with a commissure of the prosthetic valve when the prosthetic valve is arranged on the support surface.

### CROSS-REFERENCE TO RELATED APPLICATIONS

This application is a continuation of International Application No. PCT/US 2024/042418, filed Aug. 15, 2024, which claims the benefit of U.S. Patent Application No. 63/533,555, filed Aug. 18, 2023, the entire contents all of which are incorporated by reference for all purposes.

### FIELD

The present disclosure relates to apparatuses, systems, and methods for crimping a prosthetic implant on a delivery apparatus.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size. Mounting and crimping a prosthetic heart valve on a delivery apparatus involves complex steps and requires specialized skills. Accordingly, improvements to systems and methods to facilitate such mounting and crimping operations are desirable.

### SUMMARY

Described herein are alignment devices, assemblies, and methods for aligning a prosthetic heart valve in a desired circumferential and/or axial orientation within a crimping device and crimping the prosthetic heart valve onto a delivery apparatus in a desired orientation. The prosthetic heart valves delivered with the delivery apparatuses disclosed herein are, for example, radially expandable from a radially compressed state mounted on the delivery apparatus to a radially expanded state for implantation using an inflatable balloon (or an equivalent expansion device) of the delivery apparatus. Although the devices and methods disclosed herein are particularly described with respect to crimping and implanting prosthetic heart valves (for example, a prosthetic aortic valve or prosthetic mitral valve), the disclosed devices and methods can be used for crimping and implanting other types of prosthetic valves (for example, prosthetic venous valves) and/or other types of expandable prosthetic devices adapted to be implanted in various body lumens (e.g., stents, grafts, etc.).

An alignment device for a prosthetic valve can comprise a coupling portion and a support portion that tapers from a wider end that is disposed adjacent a first end of the coupling portion to a narrower end, wherein the support portion comprises an outward facing support surface that is configured to receive the prosthetic heart valve thereon.

In some examples, the coupling portion of the alignment device can comprise a coupling member configured to couple with a crimping device.

In some examples, the alignment device can comprise a central channel extending through the coupling portion and the support portion, the central channel configured to receive a delivery apparatus for the prosthetic heart valve therethrough.

In some examples, the coupling portion of the alignment device can comprise a cylindrical wall and a retaining member disposed in the cylindrical wall, wherein the retaining member is configured to retain an axial position of the alignment device inside a channel of a crimping device.

In some examples, the alignment device can comprise one or more axially extending alignment members, each alignment member extending axially outward from the first end of the coupling portion, over the support portion and to the support surface.

In some examples, each alignment member can comprise a free end configured to abut an end of the prosthetic heart valve and be axially aligned with a commissure of the prosthetic heart valve when the prosthetic heart valve is arranged on the support surface.

In some examples, the coupling portion, support portion, and axially extending alignment member of the alignment device are formed together as a unitary component.

In some examples, the coupling portion and the support portion are separate parts that are configured to be coupled to one another, and wherein the one or more alignment members are molded together with the coupling portion to form a unitary component.

In some examples, the coupling portion, including the one or more alignment members, can comprise a lower durometer material and the support portion can comprise a higher durometer material.

In some examples, the support portion can comprise a plurality of tabs that are configured to couple to the first end of the coupling portion such that the support portion and the coupling portion are coupled together.

In some examples, an alignment device for a prosthetic heart valve comprises a coupling portion comprising a coupling member configured to couple with a crimping device; a support portion that tapers from a wider end that is disposed adjacent a first end of the coupling portion to a narrower end, wherein the support portion comprises an outward facing support surface that is configured to receive the prosthetic heart valve thereon; one or more axially extending alignment members, each alignment member extending axially outward from the first end of the coupling portion, over the support portion and to the support surface, each alignment member comprising a free end configured to abut an end of the prosthetic heart valve and be axially aligned with a commissure of the prosthetic heart valve when the prosthetic heart valve is arranged on the support surface; and a central channel extending through the coupling portion and the support portion, the central channel configured to receive a delivery apparatus for the prosthetic heart valve therethrough.

In some examples, an alignment device for a prosthetic heart valve comprises a coupling portion comprising a cylindrical wall with a first end and a second end, and a retaining member disposed in the cylindrical wall, wherein the retaining member is configured to retain an axial position of the alignment device inside a channel of a crimping device; a support portion that tapers from a wider end disposed at the second end of the coupling portion to a narrower end, wherein the support portion comprises an outward facing support surface that is configured to receive the prosthetic heart valve thereon; an axially extending alignment member, the alignment member extending axially outward from the second end of the cylindrical wall of the coupling portion, over the support portion and to the support surface, wherein the alignment member is configured to abut an end of the prosthetic heart valve and be axially aligned with a specified feature of the prosthetic heart valve when the prosthetic heart valve is arranged on the support surface; and a central channel extending through the alignment device, the central channel configured to receive a delivery apparatus for the prosthetic heart valve therethrough.

In some examples, an alignment device for a prosthetic heart valve comprises a coupling portion comprising: a cylindrical wall with a first end and a second end, wherein the second end comprises an axial wall, and wherein a plurality of indentations is disposed in a radially inward facing surface of the axial wall; a coupling member extending axially from the first end toward the second end of the cylindrical wall, wherein the coupling member is configured to couple with a crimping device; and one or more axially extending alignment members, each alignment member extending axially outward from the second end of the cylindrical wall. The alignment device further comprises a support portion that tapers from a wider, first end to a narrower, second end, wherein the support portion comprises: an outward facing support surface that is configured to receive the prosthetic heart valve thereon, wherein the one or more axially extending alignment members extend over the support portion and to the support surface, each alignment member comprising a free end configured to abut an end of the prosthetic heart valve and be axially aligned with a commissure of the prosthetic heart valve when the prosthetic heart valve is arranged on the support surface; and a plurality of tabs extending axially away from the first end of the support portion, wherein the plurality of tabs is configured to couple to the plurality of indentations such that the coupling portion and support portion are coupled to one another. The alignment device further comprises a central channel extending through the coupling portion and the support portion, the central channel configured to receive a delivery apparatus for the prosthetic heart valve therethrough.

In some examples, an alignment device for a prosthetic heart valve comprises a coupling portion comprising a cylindrical wall with a first end and a second end, and a coupling member configured to couple with a crimping device, wherein the coupling member extends axially outward from the first end toward the second, and a support portion that tapers radially inward from a wider end that is disposed adjacent the second end of the coupling portion to a narrower end. The support portion comprises an outward facing support surface that is configured to receive the prosthetic heart valve thereon, and a plurality of alignment indicators spaced circumferentially apart from one another at the narrower end of the support portion, where each alignment indicator is configured to be axially aligned with a commissure of the prosthetic heart valve when the prosthetic heart valve is arranged on the support surface. The alignment device further comprises a central channel extending through the coupling portion and the support portion, the central channel configured to receive a delivery apparatus for the prosthetic heart valve therethrough.

In some examples, an alignment device comprises one or more of the components recited in Examples 1-35 and 48-92 below.

A method can comprise placing a prosthetic valve in a radially expanded configuration over a support surface of a tapered support portion of an alignment device and rotationally aligning the prosthetic valve on the support surface such that specified features of the prosthetic valve are axially aligned with alignment members of the alignment device, inserting the prosthetic valve placed over the support surface into a channel of a crimping device, and radially compressing the prosthetic valve placed within the channel by actuating the crimping device. The radially compressing causes the alignment device to be moved axially out of and away from the prosthetic valve.

In some examples, the specified features of the prosthetic valve can be commissures of the prosthetic valve.

In some examples, the support portion extends from a proximal end of a coupling portion of the alignment device, the alignment members extend axially outward from the proximal end of the coupling portion, over the support portion, and to the support surface, and the coupling portion includes a coupling member that extends axially outward from a distal end of the coupling portion toward the support portion.

In some examples, the method can comprise inserting the coupling member into a complementary recess in a distal face of the crimping device such that the alignment device is held in a specified circumferential orientation relative to the crimping device.

In some examples, a method comprises placing a prosthetic valve in a radially expanded configuration over a support surface of a tapered support portion of an alignment device and rotationally aligning the prosthetic valve on the support surface such that commissures of the prosthetic valve are axially aligned with alignment members of the alignment device, wherein the support portion extends from a proximal end of a coupling portion of the alignment device, wherein the alignment members extend axially outward from the proximal end of the coupling portion, over the support portion, and to the support surface, and wherein the coupling portion includes a coupling member that extends axially outward from a distal end of the coupling portion toward the support portion. The method further comprises inserting the prosthetic valve placed over the support surface into a channel of a crimping device, the channel being surrounded by a plurality of pressing surfaces of the crimping device; inserting the coupling member into a complementary recess in a distal face of the crimping device such that the alignment device is held in a specified circumferential orientation relative to the crimping device; and radially compressing the prosthetic valve placed within the channel by actuating the crimping device to move the pressing surfaces radially inwardly. The radially compressing causes the alignment members to deflect radially inward and the alignment device to be moved axially out of and away from the prosthetic valve.

In some examples, a method comprises one or more of the features recited in Examples 36-47 and 93 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to an example.

FIG. 1B is a perspective view of the prosthetic valve of FIG. 1A with the components on the outside of the frame shown in transparent lines for purpose of illustration.

FIG. 2A is a side view of an exemplary delivery apparatus configured to deliver and implant a radially expandable prosthetic heart valve at an implantation site.

FIG. 2B is a cross-sectional side view of a distal end portion of the delivery apparatus of FIG. 2A.

FIG. 3A is a rear perspective view of an exemplary crimping device configured to crimp a prosthetic valve onto a portion of a delivery apparatus.

FIG. 3B is a front perspective view of the crimping device of FIG. 3A.

FIG. 4 is a perspective view of an alignment device for crimping a prosthetic valve onto a valve mounting portion of a delivery apparatus, in a specified circumferential orientation and/or axial orientation.

FIG. 5A is a first cross-sectional side view of the alignment device of FIG. 4.

FIG. 5B is a second cross-sectional side view of the alignment device of FIG. 4.

FIG. 6 depicts a prosthetic valve mounted on a support portion of the alignment device of FIG. 4 with its commissures axially aligned with alignment members of the alignment device.

FIG. 7 depicts the alignment device and prosthetic valve of FIG. 6 being inserted into a first side of a crimping device.

FIG. 8 depicts the alignment device inserted into the crimping device of FIG. 7, with a coupling member and retaining member of the alignment device coupled with respective mating components of the crimping device such that the prosthetic valve is held within the crimping device in a specified circumferential orientation and axial orientation.

FIG. 9 is a cross-sectional view of the crimping device of FIG. 8 showing the coupling member of the alignment device coupled to a complementary recess of the crimping device.

FIG. 10A is a cross-sectional view of the crimping device of FIG. 9 showing the retaining member of the alignment device coupled with a complementary slot in the crimping device.

FIG. 10B is a detail view of a portion of the cross-sectional view of FIG. 10A.

FIG. 11 is a flow chart of a method for radially compressing a prosthetic valve onto a delivery apparatus with a crimping device while the prosthetic valve is held in a predetermined circumferential orientation and axial position within and relative to the crimping device.

FIG. 12 is an exploded perspective view of a two-part alignment device for crimping a prosthetic valve onto a valve mounting portion of a delivery apparatus, in a specified circumferential orientation and/or axial orientation, the two-part alignment device comprising a coupling portion and support portion that are configured to be coupled to one another.

FIG. 13 is an end view of the two-part alignment device of FIG. 12 with the coupling portion and support portion coupled to one another.

FIG. 14A is a first cross-sectional side view of the assembled two-part alignment device of FIG. 12.

FIG. 14B is a second cross-sectional side view of the assembled two-part alignment device of FIG. 12.

FIG. 15 is a perspective view of the support portion of the two-part alignment device of FIG. 12.

FIG. 16 is a perspective view of the coupling portion of the two-part alignment device of FIG. 12.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can be mounted onto a delivery apparatus in a crimped or radially compressed state for delivery to a target implantation site using the delivery apparatus. However, mounting and crimping a prosthetic heart valve on a delivery apparatus involves complex steps and requires specialized skills. For example, it may be desirable to crimp the prosthetic heart valve onto a delivery apparatus in a specified circumferential orientation relative to the delivery apparatus (e.g., such as a commissure of the prosthetic heart valve being aligned with a radiopaque marker on the delivery apparatus). In some examples, it may be desirable to crimp the prosthetic heart valve onto the delivery apparatus in a specified axial orientation on the delivery apparatus (e.g., such as on a valve mounting portion of the delivery apparatus, adjacent to a balloon shoulder of the delivery apparatus). However, when crimping using a crimping device, the prosthetic valve may be inadvertently shifted axially or rotated from its specified alignment with the delivery apparatus inserted inside the crimping device. As a result, the prosthetic heart valve may not be crimped into the specified circumferential and/or axial orientation on the delivery apparatus. This can result in the prosthetic heart valve being implanted at the native anatomy in a position that is different than desired.

In some examples, an alignment device can be used to mount a prosthetic heart valve thereon prior to crimping the prosthetic heart valve to the delivery apparatus. The alignment device can be configured with one or more alignment members that are configured to be aligned with target features on the prosthetic heart valve (e.g., one or more commissures of the prosthetic heart valve). As a result, the prosthetic heart valve can be aligned on the alignment device in a specified circumferential (or rotational) position relative to the alignment device. The alignment device can also include a coupling member that is configured to couple with a crimping device. As a result, the alignment device can be coupled with the crimping device in a specified circumferential orientation relative to the crimping device, thereby aligning the prosthetic heart valve mounted thereon in a specified circumferential orientation with the crimping device. As a result, the prosthetic heart valve can be crimped onto a delivery apparatus using the crimping device in a desired circumferential and axial orientation relative to the delivery apparatus.

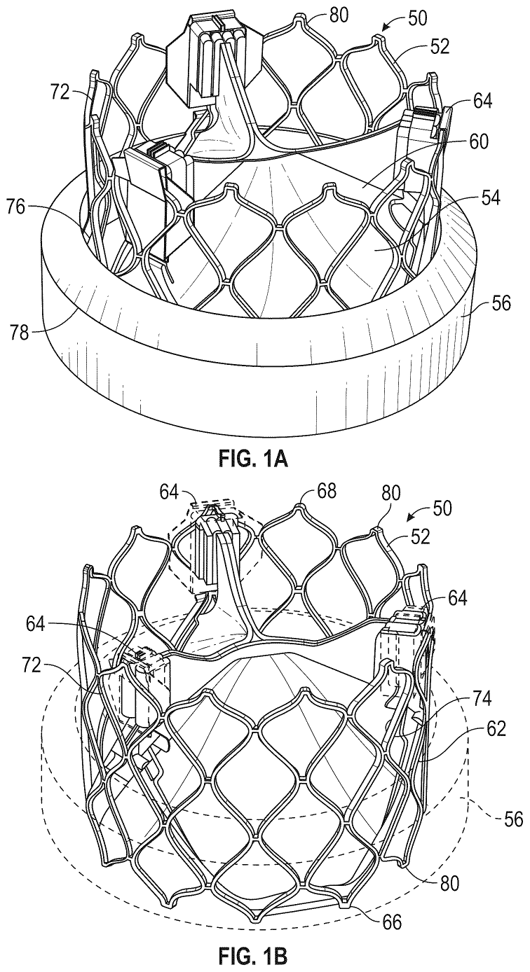

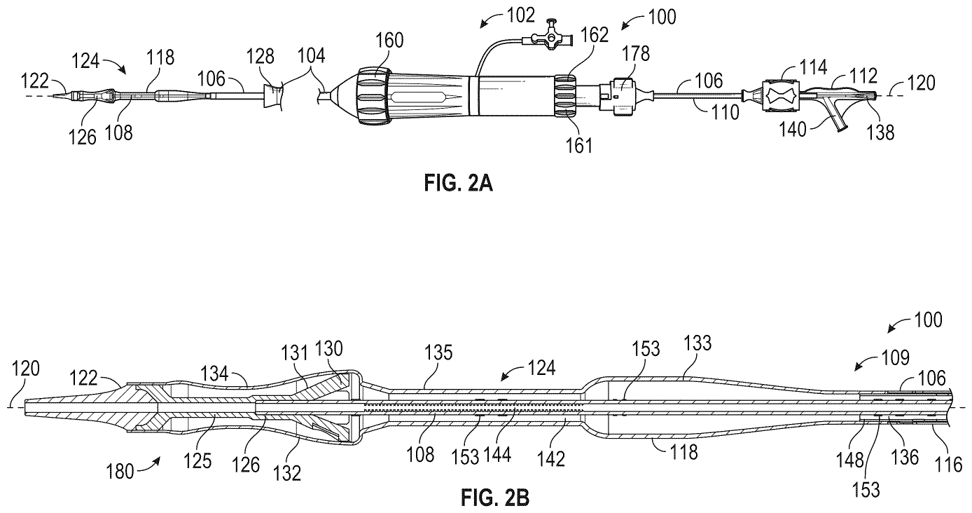

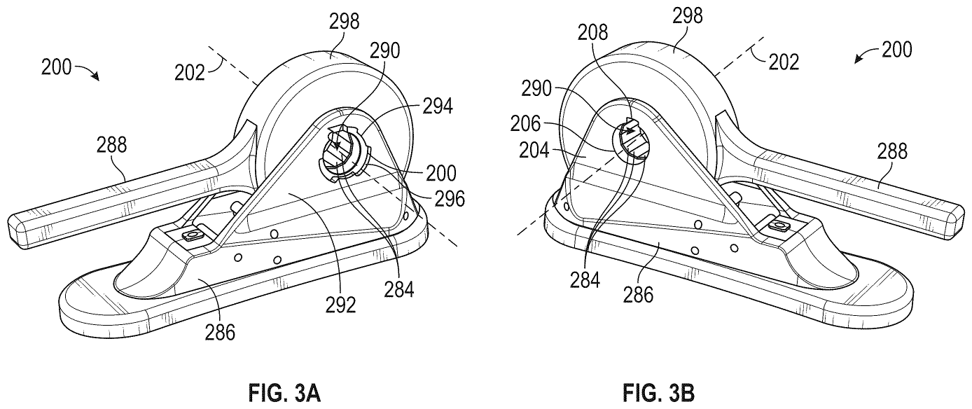

FIGS. 1A and 1B show an exemplary prosthetic heart valve in a radially expanded configuration that can be crimped into a radially compressed configuration onto a delivery apparatus (e.g., the delivery apparatus of FIGS. 2A and 2B) using a crimping device (e.g., the crimping device of FIGS. 3A and 3B).

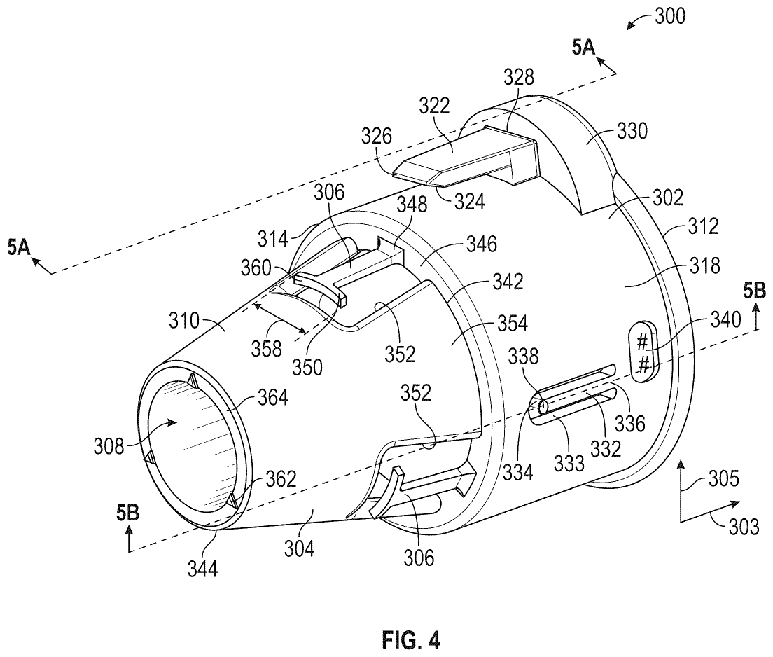

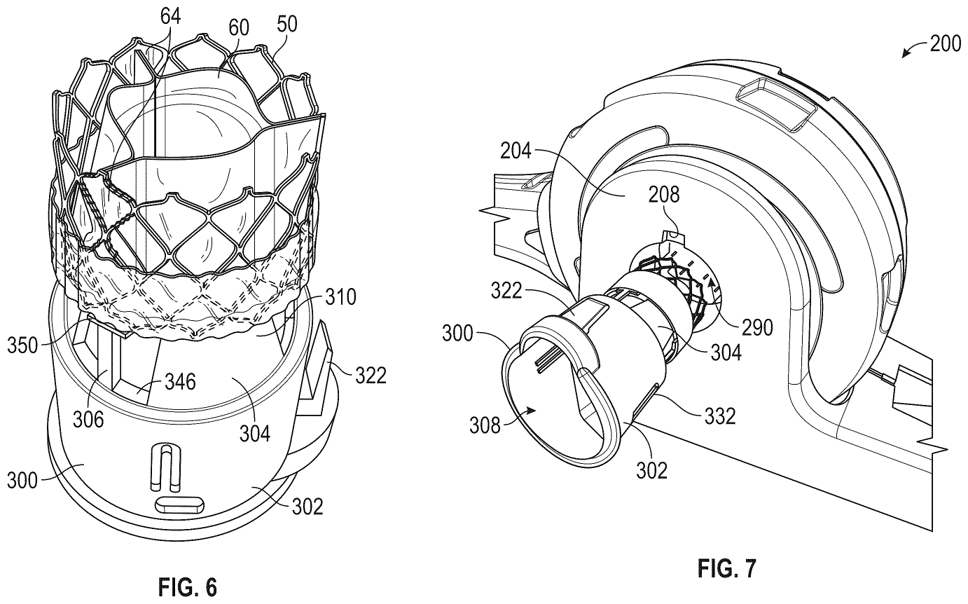

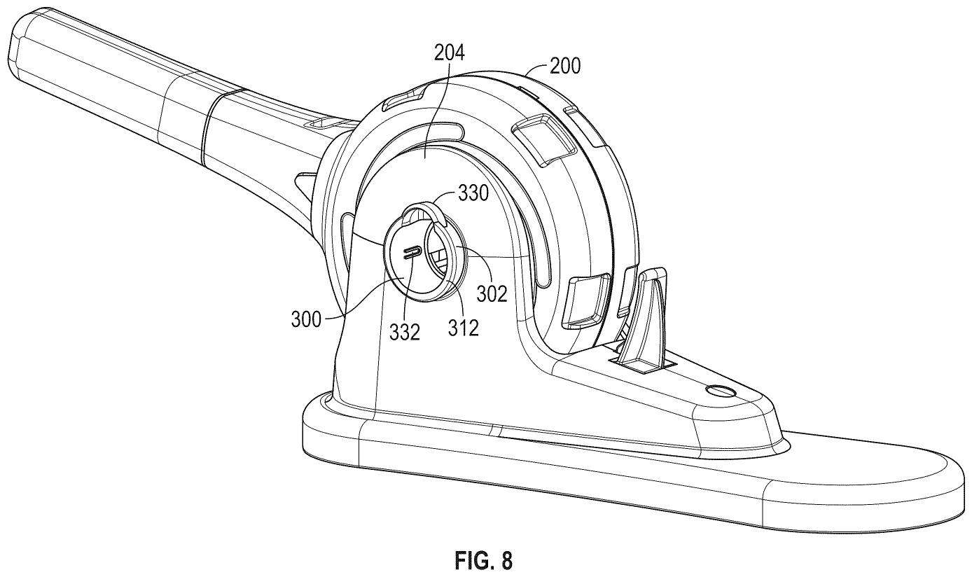

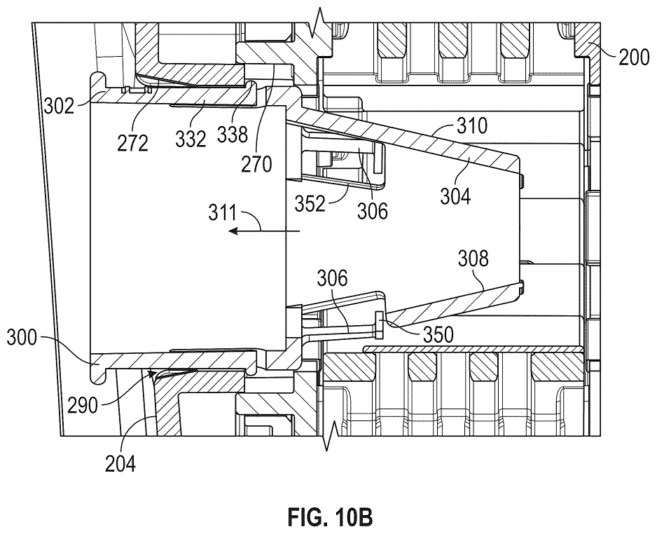

FIGS. 4-6 depict an exemplary alignment device that can be used to align the radially expanded prosthetic heart valve in a specified circumferential and axial orientation when coupled to the crimping device (FIGS. 7 and 8). For example, the prosthetic heart valve can be mounted on the alignment device such that its commissures are aligned with alignment members of the alignment device (FIG. 6). The alignment device can then be inserted into a first side of the crimping device such that an axially extending coupling member of the alignment device extends into and couples with a complementary recess in the first side of the crimping device (FIGS. 7-9). The alignment device can further comprise one or more retaining members that are configured to couple and/or snap into a slot or groove inside the crimping device, thereby axially aligning the alignment device in a specified orientation within the crimping device (FIGS. 10A and 10B). As a result, upon crimping the prosthetic heart valve onto a delivery apparatus inserted into the crimping device, the prosthetic heart valve can be mounted onto the delivery apparatus with a specified circumferential and axial orientation relative to the delivery apparatus.

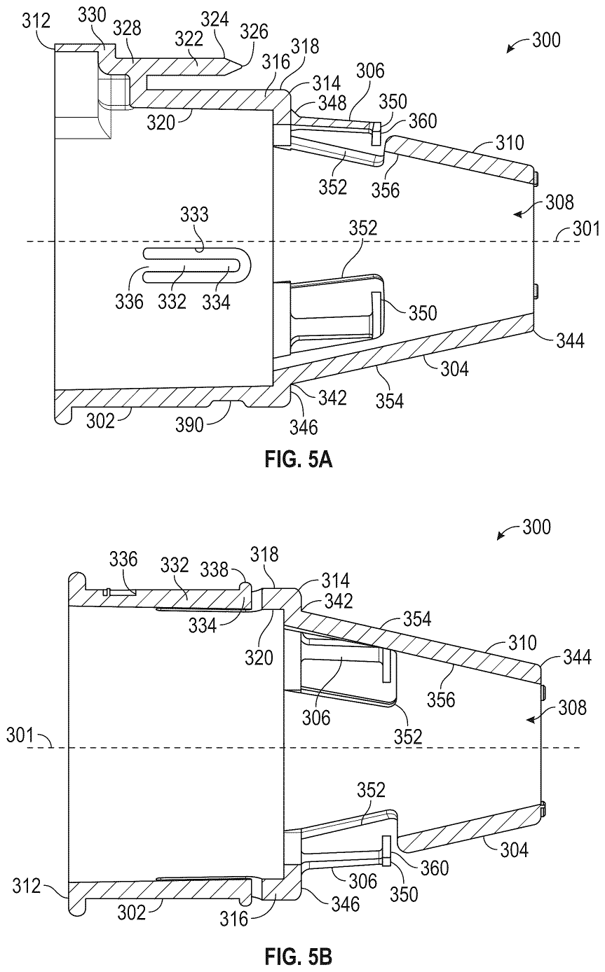

In some examples, the alignment device can be an integrated alignment device that is formed (e.g., molded) as one piece (or one unitary component) (FIGS. 4-5B).

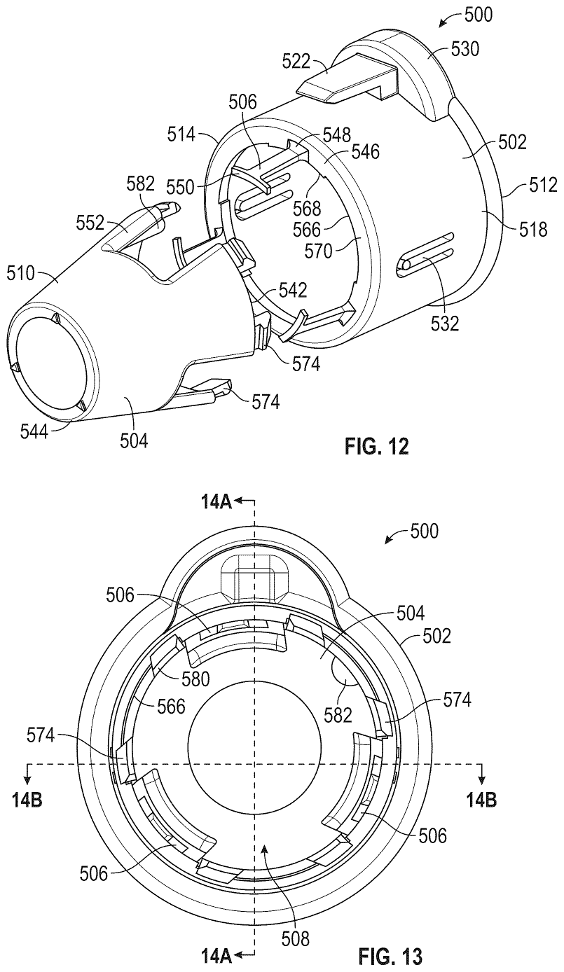

In some examples, the alignment device can be a two-part alignment device (FIGS. 12-16) where the coupling portion, which includes the alignment members, and the support portion of the alignment device comprise different material and are configured to couple to one another.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIGS. 1A and 1B show an exemplary prosthetic valve 50, according to an example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some example, the disclosed prosthetic valves can be implanted within a docking device implanted within or at the native mitral valve, such as disclosed in PCT Publication No. WO2020/247907, which is incorporated herein by reference. In some example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 50 can have three main components: a stent or frame 52, a valvular structure 54, and a sealing member 56 (e.g., fabric and/or polymeric material circumferentially positioned about at least a portion of the frame 52), (see e.g., FIG. 1A). FIG. 1B is a perspective view of the prosthetic valve 50 with the components on the outside of the frame 52 (including the sealing member 56) shown in transparent lines for purposes of illustration. The prosthetic valve 50 can have an inflow end 66 and an outflow end 68.

The valvular structure 54 can comprise three leaflets 60, collectively forming a leaflet structure, which can be arranged to collapse in a tricuspid arrangement, although in some examples there can be greater or fewer number of leaflets (e.g., one or more leaflets 60). In some examples, the leaflets 60 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

Each leaflet 60 can be coupled to the frame 52 along its inflow edge 62 (the lower edge in the figures; also referred to as “cusp edges”) and at commissures 64 of the valvular structure 54 where adjacent portions (e.g., commissure tabs) of two leaflets are connected to each other. In some examples, the commissures 64 can comprise an attachment member (e.g., comprising fabric, flexible polymer, or the like) arranged across a cell (e.g., commissure cell) of the frame 52, the cell formed by struts of the frame. The attachment member can be secured to the struts of the frame forming the cell and the adjacent portions of the two leaflets can be connected to the attachment member to form the commissure 64 (e.g., as shown in FIGS. 3-8, as described further below).

In some examples, a reinforcing element or connecting skirt, such as a fabric strip, can be connected directly to the cusp edges of the leaflets and to the struts of the frame to couple the cusp edges of the leaflets to the frame.

The frame 52 can be made of any of various suitable plastically-expandable materials (e.g., stainless steel, etc.) or self-expanding materials (e.g., Nitinol). When constructed of a plastically-expandable material, the frame 52 (and thus the prosthetic valve 50) can be crimped to a radially collapsed configuration on a delivery apparatus (e.g., catheter) and then expanded inside a patient by an inflatable balloon or equivalent expansion mechanism. Various crimping devices can be used to crimp the prosthetic valve 50 and the other prosthetic valves described herein around the delivery apparatus, such as the crimping devices described in U.S. Pat. No. 7,530,253, which is incorporated herein by reference.

In some instances, the prosthetic valve 50 can be crimped directly onto the inflatable balloon of the delivery apparatus, such that the prosthetic valve 50 is axially aligned with and disposed radially outward of the balloon during advancing the prosthetic valve on the delivery apparatus to the implantation site, for example as described in PCT Application No. PCT/US2021/047056, which is incorporated herein by reference. In alternate instances, the prosthetic valve 50 can be crimped onto the delivery apparatus axially offset from the balloon, and then moved over the balloon at the implantation site, prior to inflation of the balloon and radial expansion of the prosthetic valve, such as described in U.S. Pat. No. 9,339,384, which is incorporated herein by reference.

When constructed of a self-expandable material, the frame 52 (and thus the prosthetic valve 50) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frame 52 include, metal alloys, polymers, or combinations thereof. Example metal alloys can comprise one or more of the following: nickel, cobalt, chromium, molybdenum, titanium, or other biocompatible metal. In some examples, the frame 52 can comprise stainless steel. In some examples, the frame 52 can comprise cobalt-chromium. In some examples, the frame 52 can comprise nickel-cobalt-chromium. In some examples, the frame 52 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 52 in the illustrated example comprises a plurality of circumferentially extending rows of angled struts 72 defining rows of open cells 74 (or openings) of the frame. The frame 52 can have a cylindrical or substantially cylindrical shape having a constant diameter from the inflow end 66 to the outflow end 68 of the frame 52 as shown, or the frame 52 can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The sealing member 56 in the illustrated example is mounted on the outside of the frame 52 and functions to create a seal against the surrounding tissue (e.g., the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In some examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient's body, the outer layer 78 can expand away from the inner layer 76 to create a space between the two layers. Thus, when implanted inside the body, this allows the outer layer 78 to expand into contact with the surrounding tissue.

Additional details regarding the prosthetic valve 50 and its various components are described in U.S. Patent Publication No. 2018/0028310, which is incorporated herein by reference.

FIG. 2A shows an example delivery apparatus 100, which can be used to implant an expandable prosthetic heart valve (for example, the prosthetic valve 50 of FIG. 1A), or another type of expandable prosthetic medical device (such as a stent). A distal end portion 109 of the delivery apparatus 100 is shown in FIG. 2B. In some examples, the delivery apparatus 100 is specifically usable or adapted for use in introducing a prosthetic valve into a heart. As described further below, the delivery apparatus 100 can be configured to rotate the prosthetic valve, mounted on the delivery apparatus in a radially compressed state, at the target implantation site (for example, at a native valve of the heart) to achieve commissure alignment between the native valve and prosthetic valve after deploying the prosthetic valve.

In the depicted example, the delivery apparatus 100 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends both proximally and distally from the handle 102. The portion of the intermediate shaft 106 extending distally from the handle 102 also extends coaxially through the outer shaft 104. Additionally, the delivery apparatus 100 can further comprise an inner shaft 108 extending distally from the handle 102 and coaxially through the intermediate shaft 106 and the outer shaft 104. The inner shaft 108 also extends proximally from the handle 102 and coaxially through the intermediate shaft 106.

The outer shaft 104 and the intermediate shaft 106 are configured to translate longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110. The knob 114 can be configured to rotate the intermediate shaft 106 around the central longitudinal axis 120 of the delivery apparatus 100 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion 116 that extends distally beyond a distal end of the outer shaft 104 when the distal end of the outer shaft 104 is positioned away from an inflatable balloon 118 of the delivery apparatus. A distal end portion of the inner shaft 108 can extend distally beyond the distal end portion 116 of the intermediate shaft 106.

The balloon 118 can be coupled to the distal end portion 116 of the intermediate shaft 106. For example, a proximal end portion of the balloon 118 can be coupled to and/or around a distal end 148 of the intermediate shaft 106.

The balloon 118 can comprise a distal end portion (or section) 132, a proximal end portion (or section) 133, and an intermediate portion (or section) 135, the intermediate portion 135 disposed between the distal end portion 132 and the proximal end portion 133.

In some examples, a distal end of the distal end portion 132 of the balloon 118 can be coupled to a distal end of the delivery apparatus 100, such as to a nose cone 122, or to an alternate component at the distal end of the delivery apparatus 100 (for example, a distal shoulder). In some examples, the intermediate portion 135 of the balloon 118 can overlay a valve mounting portion 124 of a distal end portion 109 of the delivery apparatus 100, the distal end portion 132 can overly a distal shoulder 126 of the delivery apparatus 100, and the proximal end portion 133 can surround a portion of the inner shaft 108 (FIG. 2B). The valve mounting portion 124 and the intermediate portion 135 of the balloon 118 can be configured to receive a prosthetic valve in a radially compressed state.

As described herein, rotation of the intermediate shaft 106 can cause rotation of the balloon 118 and the prosthetic valve mounted thereon for rotational positioning of the prosthetic valve relative to the native anatomy at the target implantation site.

The delivery apparatus 100 can include a balloon shoulder assembly 180 configured to maintain the prosthetic heart valve or other medical device at a fixed position on the balloon 118 during delivery through the patient's vasculature. The balloon shoulder assembly 180 can include a distal shoulder 126 arranged within a distal end portion of the balloon 118 and coupled to the distal end portion of the inner shaft 108. The distal shoulder 126 can be configured to resist movement of the prosthetic valve or other medical device mounted on the valve mounting portion 124 distally, in an axial direction (for example, along the central longitudinal axis 120), relative to the balloon 118.

For example, the distal shoulder 126 can include a flared portion 131 arranged adjacent to the valve mounting portion 124. In some examples, the flared portion 131 can include a plurality of wings 130 that flare radially outward from a base portion 125 (for example, shaft) of the distal shoulder 126, toward the valve mounting portion 124.

The outer shaft 104 can include a distal tip portion 128 mounted on its distal end. In some examples, the distal tip portion 128 can be configured as a flex adaptor including a plurality of inner and outer helical grooves. The outer shaft 104 and the intermediate shaft 106 can be translated axially relative to one another to position the distal tip portion 128 adjacent to a proximal end of the valve mounting portion 124, when a prosthetic valve is mounted in the radially compressed state on the valve mounting portion 124 and during delivery of the prosthetic valve to the target implantation site. As such, the distal tip portion 128 can be configured to resist movement of the prosthetic valve relative to the balloon 118 proximally, in the axial direction, relative to the balloon 118, when the distal tip portion 128 is arranged adjacent to a proximal side of the valve mounting portion 124.

In some examples, the nose cone 122 can be disposed distal to and be coupled to the distal shoulder 126. In some examples, the nose cone 122 can be coupled to the distal end portion of the inner shaft 108.

In some examples, the delivery apparatus 100 can comprise one or more markers or marker bands 153 that are configured to indicate to a user a location of a specified component of the delivery apparatus. In some examples, the one or more marker bands 153 can be radiopaque. In some examples, one or more marker bands 153 can be radially compressed (for example, crimped) onto the inner shaft 108.

In some examples, the distal end portion 132 of the balloon 118 can include a radial depression 134 that is depressed radially inwardly, toward the central longitudinal axis 120, relative to an outermost radial surface of the distal shoulder 126 and an outermost radial surface of the nose cone 122.

An annular space 136 can be defined between an outer surface of the inner shaft 108 and an inner surface of the intermediate shaft 106. In some examples, the annular space 136 can be referred to as an inner lumen of the intermediate shaft 106. In some examples, the annular space 136 can be configured to receive an inflation fluid from a fluid source via the second port 140 of the adaptor 112 (for example, the annular space 136 can be in fluid communication with the second port 140 of the adaptor 112). The annular space 136 can be fluidly coupled to a fluid passageway 142 formed between the outer surface of the distal end portion of the inner shaft 108 and an inner surface of the balloon 118. As such, fluid from the fluid source can flow to the fluid passageway 142 from the annular space 136 to inflate the balloon 118 and radially expand and deploy the prosthetic valve.

In some examples, after crimping of the prosthetic valve onto the valve mounting portion 124, the distal tip portion 128 can be advanced over the proximal end portion 133 of the balloon 118. As a result, fluid arranged within the proximal end portion 133 of the balloon 118 can be displaced and pushed distally, within the balloon 118, to the distal end portion 132 of the balloon 118. The radially depressed, distal end portion 132 of the balloon 118 can then radially expand (for example, inflate partially) as it receives the displaced fluid to an expanded state. The radial depression 134 can be configured (for example, sized) so that the distal end portion 132 can receive the displaced fluid without radial expanding the portion of the balloon 118 within the valve mounting portion 124, thereby preventing the crimped profile of the prosthetic valve from increasing.

An inner lumen 144 of the inner shaft 108 can be configured to receive a guidewire therethrough, for navigating the distal end portion 109 of the delivery apparatus 100 to the target implantation site. As introduced above, the first port 138 of the adaptor 112 can be coupled to the inner lumen 144 and configured to receive the guidewire. For example, the distal end portion 109 of the delivery apparatus 100 can be advanced over the guidewire, to the target implantation site.

As shown in FIG. 2A, the handle 102 can include a steering mechanism configured to adjust the curvature of the distal end portion 109 of the delivery apparatus 100. For example, the handle 102 can include an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion 109 of the delivery apparatus 100. Further details on steering or flex mechanisms for the delivery apparatus are described in U.S. Pat. No. 9,339,384, which is incorporated by reference herein in its entirety.

The handle 102 can further include an adjustment mechanism 161 including an adjustment member, such as the illustrated rotatable knob 162. The adjustment mechanism 161 can be configured to move (thus adjust the axial position) of the intermediate shaft 106 relative to the outer shaft 104. The handle 102 can also include a locking mechanism configured to retain (for example, lock) the position of the intermediate shaft 106 relative to the handle 102. In some examples, the locking mechanism can include another adjustment member, which can be configured as a rotatable knob 178. In some examples, rotating the knob 178 to a locked position can cause the intermediate shaft 106 to frictionally engage with other components of the handle 102, thereby restraining movement of the intermediate shaft 106 for fine positioning of the prosthetic valve mounted on the distal end portion of the delivery apparatus 100. Rotating the knob 178 to an unlocked position allows axial and rotational movement of the intermediate shaft 106 relative to the proximal end portion of the handle 102. For example, rotation of the knob 162 can cause the intermediate shaft 106 to move axially relative to the outer shaft 104 (either in the proximal or distal direction, depending on the direction the knob 162 is rotated).

Further details on the adjustment mechanism and locking mechanism of the handle 102 can be found in U.S. Pat. No. 9,339,384, which is incorporated by reference herein in its entirety. Additional examples of delivery apparatuses and related steering mechanism can be found in U.S. Pat. No. 8,568,472, which is incorporated herein by reference in its entirety.

According to certain examples, to implant a prosthetic valve (for example, prosthetic valve 50) in a native heart valve of the patient, the delivery apparatus 100 can be introduced into vasculature of the patient. The prosthetic valve can be initially retained in a radially compressed configuration on the valve mounting portion 124 (and over the balloon 118) of the delivery apparatus 100.

In some instances, once inside the patient's vasculature, the position (e.g., axial position) of the prosthetic valve relative to the balloon 118 can be adjusted such that the prosthetic valve 50 is centered on the balloon 118. In some instances, the axial position of the prosthetic valve relative to the balloon 118 may not be adjusted.

When navigating the prosthetic valve through an arched region of the vasculature (for example, the aortic arch), the curvature of the distal end portion 109 of the delivery apparatus 100 can be adjusted, for example, by rotating the knob 160 to increase or decrease the tension in the pull wire which extends between the handle 102 and the distal end of the outer shaft 104. The prosthetic valve can be positioned within or adjacent an annulus of the native heart valve. Prior to inflating the balloon 118, the outer shaft 104 can be retracted proximally away from the balloon 118 for a sufficient distance so that the outer shaft does not interfere with balloon inflation. This can be accomplished, for example, by holding the adaptor 112 stationary against the operating table and rotating the knob 162 in a direction that causes the handle 102 and the outer shaft 104 to move proximally away from the balloon 118. Then, the prosthetic valve can be radially expanded and deployed by inflating the balloon 118. Inflation of the balloon 118 can radially expand the prosthetic valve 50 so that the prosthetic valve 50 contacts the native annulus. The expanded prosthetic valve 50 becomes anchored within the native aortic annulus by the radial outward force of the valve's frame against the surrounding tissue.

As described above, the knob 114 of the handle 102 can be configured to rotate the intermediate shaft 106, thereby rotating the balloon 118 mounted on the intermediate shaft 106 and a radially compressed prosthetic valve mounted on the balloon 118, around the valve mounting portion 124. Thus, rotating the knob 114 can rotate the prosthetic valve, around the central longitudinal axis 120, into a desired (circumferential or rotational) orientation relative to the native anatomy at the target implantation site.

For example, to implant a prosthetic valve (for example, prosthetic valve 50) at a native aortic valve, instead of deploying the prosthetic valve with the delivery apparatus 100 in a random rotational orientation relative to the aorta, which may result in commissures (for example, commissures 64) of the prosthetic valve being arranged in front of the coronary arteries, it may be desirable to deploy the prosthetic valve in a targeted rotational orientation where the commissures are positioned away from and do not block the coronary arteries (e.g., to reduce the likelihood blocking coronary access during subsequent interventional procedures). Specifically, the delivery apparatus 100 can be configured to control the rotational positioning of the prosthetic heart valve relative to the native valve, to achieve the commissure alignment, thereby increasing access to the coronary arteries. Additionally, this positioning of the prosthetic heart valve can facilitate a later, leaflet cutting procedure that provides increased access to the coronary arteries.

Further details on methods of aligning commissures of native valve with commissures of prosthetic valve after deploying the prosthetic valve are described in PCT Patent Publication No. WO 2022/046585, which is incorporated by reference herein in its entirety.

FIGS. 3A-16 show examples of various components that can be used to crimp a prosthetic valve (such as prosthetic valve 50) onto a valve mounting portion of a delivery apparatus (for example, the valve mounting portion 124 of delivery apparatus 100) in a predetermined position and orientation relative to the delivery apparatus. The prosthetic valve may be crimped to the valve mounting portion of the delivery apparatus in a variety of manners. In some examples, a crimping device, such as the crimping device 200 shown in FIGS. 3A and 3B, can be used to crimp the prosthetic valve onto the valve mounting portion of the delivery apparatus. As described further below, the crimping device 200 can include mating interfaces, on opposite sides of the crimping device 200, that are configured to receive and mate with corresponding mating interfaces on first and second components of the mounting assembly.

FIG. 3A illustrates a rear perspective view of the crimping device 200 (or a view from the proximal side of the crimping device 200) and FIG. 3B illustrates a front perspective view of the crimping device 200 (or a view from the distal side of the crimping device 200). The crimping device 200 can include a base 286, an actuator in the form of a handle 288, and a channel 290 for the prosthetic valve and the delivery apparatus to be inserted into. The crimping device 200 may include a proximal face 292 including a proximal opening 294 that leads into the channel 290. The proximal opening 294 may be configured for the delivery apparatus to be inserted into the channel 290 therethrough.

In some examples, the proximal face 292 can include a mating interface with mating structures 296 in the form of cut-outs that can be configured to mate with a positioning device (for example, a positioning device coupled to a distal end portion of delivery apparatus, as disclosed in PCT Patent Publication No. WO 2022/046585, which is already incorporated by reference above). For example, the mating interface can include one or more mating structures 296.

The crimping device 200 can further include a rotatable body 298 configured to be rotated with rotation of the handle 288. The crimping device 200 may operate by a plurality of pressing surfaces 284 surrounding the channel 290 and being configured to apply a compressive force to radially compress a prosthetic valve positioned within the channel 290 (for example, prosthetic valve 50). The pressing surfaces 284 may surround an axis 202 of the channel 290. The pressing surfaces 284 may be configured such that as the rotatable body 298 is rotated, a body presses and moves the pressing surfaces 284 towards the center of the channel 290 and the diameter of the channel 290 reduces. The pressing surfaces 284 may form an iris structure that allows the pressing surfaces 284 to move towards the center of the channel 290 and reduce the diameter of the channel 290. A prosthetic valve positioned within the channel 290 will accordingly be compressed within the channel 290, due to the radially compressive force of the pressing surfaces 284 against the prosthetic valve.

As shown in FIG. 3B, the crimping device 200 may include a distal face 204 including a distal opening 206 that leads into the channel 290. The distal face 204 can include a mating interface, which can comprise a cut-out portion 208. In some examples, the cut-out portion 208 can be configured as (and referred to as) a recess, notch, indentation, depression, or the like, in the distal face 204. The cut-out portion 208 (or recess 208) can be configured (for example, shaped) to receive an alignment device of a valve support member for the prosthetic valve (for example, the coupling member 322 shown in FIGS. 4-7 or FIGS. 12-16, as described further below).

The distal opening 206 can be configured for a portion of the delivery apparatus to pass therethrough during a crimping operation being performed by the crimping device 200.

The configuration of a crimping device can be varied in alternate examples.

For crimping a prosthetic valve onto a valve mounting portion of a delivery apparatus, in a specified circumferential (or rotational) orientation and/or axial orientation (or position), an alignment device can be used. A first portion of the alignment device can be configured to receive the prosthetic valve (e.g., prosthetic valve 50) thereon and a second portion of the alignment device can couple to a crimping device (e.g., crimping device 200). For example, the alignment device can be configured to rotationally (or circumferentially) align the prosthetic valve on the first portion of the alignment device and couple with the crimping device such that the prosthetic valve is held within the crimping device in a specified circumferential orientation and/or axial orientation relative to the crimping device. As a result, the prosthetic valve can be crimped into a radially compressed configuration onto a valve mounting portion of a delivery apparatus (e.g., delivery apparatus 100) such that the radially compressed prosthetic valve is in a specified circumferential orientation and/or axial orientation relative to the delivery apparatus.

Further, in some examples, it may be desirable to maintain the leaflets (for example, leaflets 60 of prosthetic valve 50) in an open position during crimping of the prosthetic valve to the delivery apparatus, thereby reducing a likelihood of degradation to the leaflets and/or attachments of the leaflets to a frame of the prosthetic valve. Thus, in some examples, the first portion (a valve support portion) of the alignment device can be configured to support and/or maintain one or more leaflets of the prosthetic valve in an open position within the crimping device and during crimping.

FIGS. 4-10B depict an exemplary alignment device 300 that is configured to receive a prosthetic valve thereon and couple to a crimping device. As shown in the perspective view of FIG. 4 and the cross-sectional views of FIGS. 5A and 5B, the alignment device 300 includes a coupling portion 302, a support portion 304 (valve support portion), and one or more alignment members 306. The alignment device 300 also includes a central channel 308 extending through the coupling portion 302 and the support portion 304. The central channel 308 is configured to receive a delivery apparatus for the prosthetic valve therethrough (e.g., delivery apparatus 100).

As described more fully below, the coupling portion 302 can be configured to releasably couple to a crimping device, such as the crimping device 200 shown in FIGS. 3A-3B and 7-10B. The support portion 304 can be configured to be inserted into a prosthetic valve (for example, prosthetic valve 50) so that leaflets (for example, leaflets 60) of the prosthetic valve can contact and rest upon a support surface 310 of the support portion 304. The alignment members 306 can be used to space the prosthetic valve away from the coupling portion 302 and achieve a desired rotational alignment of the prosthetic valve relative to the coupling portion 302 and the crimping device.

In some examples, as shown in FIGS. 4-5B, the support portion 304 is disposed at and extends proximally from the coupling portion 302. In some instances, the support portion 304 and coupling portion 302 (and the alignment members 306) can be formed (e.g., molded together) as one piece (as a unitary component).

As introduced above, the coupling portion 302 can be configured to be inserted into the channel 290 of the crimping device 200 (from the distal side or distal face 204 of the crimping device 200) and couple to the crimping device 200.

The coupling portion 302 includes a first end 312 (distal end) and a second end 314 (proximal end).

The coupling portion 302 includes a cylindrical wall 316, which has an outer surface 318 and an inner surface 320 (FIGS. 4-5B). The inner surface 320 defines a lumen of the coupling portion 302 that forms a portion of the central channel 308. The cylindrical wall 316 extends between the first end 312 and the second end 314 of the coupling portion 302. In some examples, the first end 312 can be referred to as a first end of the cylindrical wall 316 and the second end 314 can be referred to as the second end of the cylindrical wall 316.

The coupling portion 302 includes a coupling member 322 (FIGS. 4 and 5A) that is configured to rotationally align the coupling portion 302 with the crimping device (e.g., crimping device 200, as shown in FIGS. 7-9). The coupling member 322 can be circumferentially positioned on the coupling portion 302, proximate to the first end 312, such that it aligns the coupling portion 302 in a predetermined circumferential orientation within the channel 290 of the crimping device 200.

The coupling member 322 is configured (shaped) to mate with the cut-out portion 208 (or recess 208) of the crimping device 200. In this manner, the coupling member 322 rotationally aligns the coupling portion 302, and thus the valve mounted on the support portion 304, with the crimping device 200. As a result of this coupling between the coupling member 322 and cut-out portion 208, the alignment device 300, and prosthetic valve mounted thereon, are prevented from rotating during crimping using the crimping device 200.

In some examples, as shown in FIGS. 4 and 5A, the coupling member 322 is an axially extending projection that extends axially away from the first end 312 toward the second end 314 of the coupling portion 302.

A free end 324 of the coupling member 322 tapers at both a top (radially outward facing side) and bottom (radially inward facing side) to a point 326 (or tapered end) (FIGS. 4 and 5A). In some instances, instead of tapering to a point 326, the free end 324 can taper to a narrower region (e.g., that is a little wider than shown in FIG. 4 and can be referred to as a tapered end). By having a taper on both sides of the free end 324 of the coupling member 322, the alignment device 300 can be easier to manufacture.

In some instances, the free end 324 may only taper on one side of the coupling member 322 (e.g., only from the top or radially outward facing side to the point).

The tapered free end 324 can allow for easier insertion into and coupling with the cut-out portion 208 in the crimping device.

In some examples, an attached end 328 of the coupling member 322 (which is opposite the free end 324) can extend or protrude outward from a base element 330 (e.g., ledge, ridge, overhang, or bump) of the coupling portion 302 (FIGS. 4 and 5A). The base element 330 can extend axially from the first end 312 and extend radially outward, away from the outer surface 318 of the cylindrical wall 316. As a result, the coupling member 322 can be spaced radially away and outward from the outer surface 318 of the cylindrical wall 316.

In some instances, as shown in FIG. 8, when the coupling member 322 is inserted into and coupled with the cut-out portion 208 of the crimping device 200, the base element 330 can be disposed against the distal face 204 of the crimping device 200 (outside the channel 290).

In some examples, the coupling member 322 can have different configurations, such as a recess, snap-in feature, or alternate alignment feature that is configured to mate with a corresponding mating interface of the crimping device 200. As one example, the crimping device can comprise a projection (in lieu of the cut-out portion 208) and the coupling member can comprise a slot (in lieu of the projection 322) configured to receive the projection of the crimping device.

Returning to FIG. 4, the cylindrical wall 316 of the coupling portion 302 can include one or more retaining members 332 which can be configured as axial locking mechanisms that prevent the coupling portion 302 from moving axially relative to the crimping device 200 after the alignment device 300 is attached to the crimping device 200. As a result, the alignment device 300 can be retained in a predetermined axial position within the crimping device 200, and relative to a delivery apparatus inserted into and coupled with the crimping device.

As shown in FIGS. 4 and 5A, the coupling portion 302 includes two retaining members 332 spaced 140-180 degrees apart from one another around the coupling portion 302.

However, in some examples, the coupling portion 302 can include more or less than two retaining members 332 (e.g., one, three, or the like).

Each retaining member 332 can comprise a finger with a free end 334 and attached end 336 that are formed by a U-shaped slot 333 in (through) the cylindrical wall 316. The free end 334 includes a protrusion 338 (or nub or bump) that protrudes radially outward from an outer surface of the free end 334.

The retaining member 332 can be configured as a cantilever, where the free end 334 deflects radially inward as the coupling portion 302 is slid into the channel 290 of the crimping device and then deflects radially outward with the protrusion 338 snapping into a complementary-shaped pocket or slot 270 in the crimping device (see FIGS. 10A and 10B).

A length of the retaining member 332 and size (e.g., radius or height) of the protrusion 338 can be specified such that the free end 334 easily snaps into the slot 270 in the crimping device 200 with little force (from the user) but is also easily released (removed from the slot 270) as the crimping device 200 is actuated (to radially compress or crimp the prosthetic valve).

In some instances, the protrusion 338 can be a spherical nub with a radius that is specified to allow easy insertion and locking into the predetermined axial position within the crimping device 200 and also easy release from the crimping device (such that the alignment device 300 can be removed) upon crimping with the crimping device 200.

In some instances, as shown in FIG. 4, the alignment device 300 can include a visual indicator 340 that enables a user to easily identify the type of alignment device 300 being used. In some examples, the visual indicator 340 is disposed in the outer surface 318 of the cylindrical wall 316 and can comprise letters or numbers (## is shown for illustration purposes only) that correspond to the type of alignment device 300 (such as a specified size out of several possible sizes, which may correspond to a size or type of prosthetic valve to be mounted onto the support portion 304).

In some examples, as shown in FIG. 5A, the cylindrical wall 316 can include a flat indentation 390 that is depressed into the outer surface 318 toward the inner surface 320. The indentation 390 can be the location of the molding gate for injection molding the alignment device 300.

As introduced above, the support portion 304 can be configured to rotationally align the prosthetic valve on a support surface 310 of the support portion 304, be inserted into the channel 290 of the crimping device 200 and receive a delivery apparatus through the central channel 308.

The support portion 304 includes a first end 342 (distal end) and second end 344 (proximal end). The first end 342 is disposed adjacent and attached to the second end 314 of the coupling portion 302.

In some instances, the first end 342 of the support portion 304 can be attached to an axial wall 346 of the coupling portion 302 (FIGS. 4-5B). The axial wall 346 can be arranged normal to a central longitudinal axis 301 of the alignment device 300 and perpendicular to the cylindrical wall 316.

The support portion 304 tapers from the first end 342 to the second end 344, such that the second end 344 is narrower (in diameter) than the first end 342. As a result, the support surface 310 which is configured to receive the prosthetic heart valve thereon, also tapers in diameter. In this way, the support surface 310 can be referred to as a conical support surface.

The alignment device 300 includes one or more axially extending alignment members 306 spaced circumferentially apart from one another around the alignment device 300. In some examples, the alignment device 300 includes three alignment members 306 (one for each commissure of the prosthetic valve). In some examples, the alignment device 300 can include more or less than three alignment members 306 (e.g., one, two, or the like).

As shown in FIGS. 4-5B, each alignment member 306 includes an attached end 348 and a free end 350 disposed axially away from the attached end 348. The attached end 348 is disposed at (coupled to or formed together with) the second end 314 of the coupling portion 302.

In some examples, the attached end 348 is disposed at (or formed with) the axial wall 346 of the coupling portion 302. Each alignment member extends axially outward from the axial wall 346, over a portion of the support portion 304 and to the support surface 310.

The support portion 304 can include one or more cut-outs or windows 352 disposed adjacent to the first end 342. In some examples, each window 352 can extend from the first end 342 to the support surface 310.

The support portion 304 can have one window 352 for each alignment member 306 (e.g., three windows 352 in the example shown in FIGS. 4-5B). As such, each window 352 can be configured to receive the free end 350 of a respective alignment member 306 therein.

Each window 352 can extend through a wall of the support portion 304, from an outer surface 354 (which is continuous with the support surface 310) to an inner surface 356 of the support portion 304. Each window 352 can have a width (in the circumferential direction) that is at least as wide as or wider than the free end 350 of the corresponding alignment member 306.

For example, the free end 350 of each alignment member 306 is configured to deflect or bend radially inward, into the corresponding window 352, in response to a radially inward directed force applied to its outer surface (e.g., during crimping with the crimping device 200).

The free end 350 of each alignment member 306 is also configured to abut an end of the prosthetic valve and be axially aligned with a commissure of the prosthetic valve, when the prosthetic valve is mounted on the support surface 310 (as shown in FIG. 6).

In some examples, as shown in FIGS. 4-5B, the alignment members 306 can be T-shaped. For example, a top or wider portion of the T shape can form the free end 350 and a shaft of the T shape can extend between the free end 350 and the attached end 348.

A width 358 of the free end 350 (FIG. 4) can be sized such that it abuts a specified portion of an end of a prosthetic valve when disposed on the support surface 310. For example, as shown in FIG. 6, the width 358 of the free end 350 can be specified such that it abuts two apices at an inflow end (or an outflow end) of the prosthetic valve, when the shaft portion of the T-shape of the alignment member 306 is aligned with a commissure 64 of the prosthetic valve.

In some instances, the width 358 of the free end 350 of the alignment member 306 can be shorter or longer than shown in FIGS. 4-6.

In some instances, the shaft of each alignment member 306 can be wider or narrower than shown in FIGS. 4-5B.

In some examples an axial face 360 of the free end 350 which is configured to abut the prosthetic valve, when the prosthetic valve is mounted on the support surface 310, is relatively planar (FIGS. 4-5B).

In some examples, the free end 350 of each alignment member 306 can curve in the circumferential direction such that it follows a curve of the support portion 304 (and a curve of the prosthetic valve when mounted on the support surface 310).

A thickness (in the radial direction) of the alignment member 306 can be specified such that the alignment member 306 can deflect into the window 352 and become aligned with the outer surface 354 during crimping, as explained further below.

For reference, a coordinate axis system is shown in FIG. 4, which depicts the radial direction 305 and axial direction 303, which are relative to a central longitudinal axis 301 of the alignment device 300.

In some examples, one or more alignment indicators 362 can be disposed in an axial wall 364 at the second end 344 of the support portion 304. Each alignment indicator 362 can be axially aligned with a corresponding alignment member 306. As such, each alignment indicator 362 can be configured to indicate an axial location for a commissure of a prosthetic valve when mounted on the support surface 310.

In some examples, the alignment indicators 362 can be arrows, lines, or variously shaped raised markings protruding from the axial wall 364.

In some examples, the alignment indicators 362 can be arrows, lines, or variously shaped markings in the axial wall 364. For example, the alignment indicators 362 can be colored differently than a remainder of the alignment device 300.

In some examples, the alignment device 300 may not include alignment indicators 362.

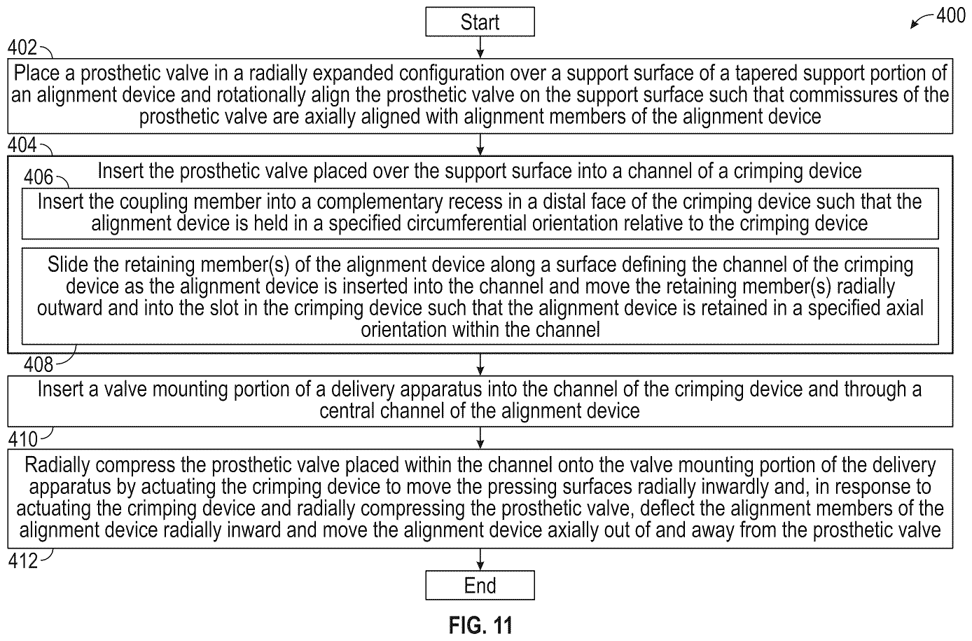

FIG. 11 is a flow chart of a method 400 for radially compressing a prosthetic valve onto a delivery apparatus with a crimping device while the prosthetic valve is held in a predetermined circumferential orientation and axial position within and relative to the crimping device. FIG. 11 is described below with reference to FIGS. 6-10B which depict using the alignment device 300 with a crimping device 200 (or another, similar crimping device) to radially compress and crimp a prosthetic valve (e.g., prosthetic valve 50) onto a valve mounting portion of a delivery apparatus (e.g., delivery apparatus 100).

However, it should be noted that the method explained below with reference to FIGS. 6-11 can also be used with other alignment devices that are configured to rotationally align commissures of a prosthetic valve relative to the crimping device and axially align the prosthetic valve within the crimping device, such as the alignment device 500 shown in FIGS. 12-16, and/or differently configured crimping devices (but which have similar mating components for mating with the alignment device, as described herein).

At 402 of FIG. 11, the method 400 begins by placing a prosthetic valve (e.g., prosthetic valve 50) in a radially expanded (or at least partially radially expanded) configuration over a support surface of a tapered support portion of an alignment device and rotationally aligning the prosthetic valve on the support surface such that commissures of the prosthetic valve are axially aligned with alignment members of the alignment device. For example, as shown in FIG. 6, the prosthetic valve 50 can be positioned on the support surface 310 of the support portion 304 of the alignment device 300. As a result, leaflets 60 of the prosthetic valve 50 can be held open. The prosthetic valve 50 can be rotationally (or circumferentially) aligned on the support surface 310 such that the commissures 64 of the prosthetic valve 50 are axially aligned with the corresponding alignment members 306.

In some examples, the prosthetic valve 50 is pushed onto the support surface 310 until the inflow end of the prosthetic valve 50 abuts the free ends 350 of the alignment members 306.

In some examples, as shown in FIG. 4, the free end 350 of each alignment member 306 abuts two apices at the inflow end of the prosthetic valve 50 which are covered by the outer skirt (or sealing member).

At 404, the method 400 includes inserting the prosthetic valve placed over the support surface into a channel of the crimping device. For example, as shown in FIG. 7, the alignment device 300, with the prosthetic valve 50 mounted thereon, can be inserted into the distal side of the crimping device 200, with the valve and the second end 344 of the support portion 304 entering the channel 290 first.

The method at 404 can include, at 406, inserting the coupling member 322 into the complementary recess (e.g., the cut-out portion 208) in the distal face of the crimping device 200 such that the alignment device 300 is held in a specified circumferential orientation relative to the crimping device 200.

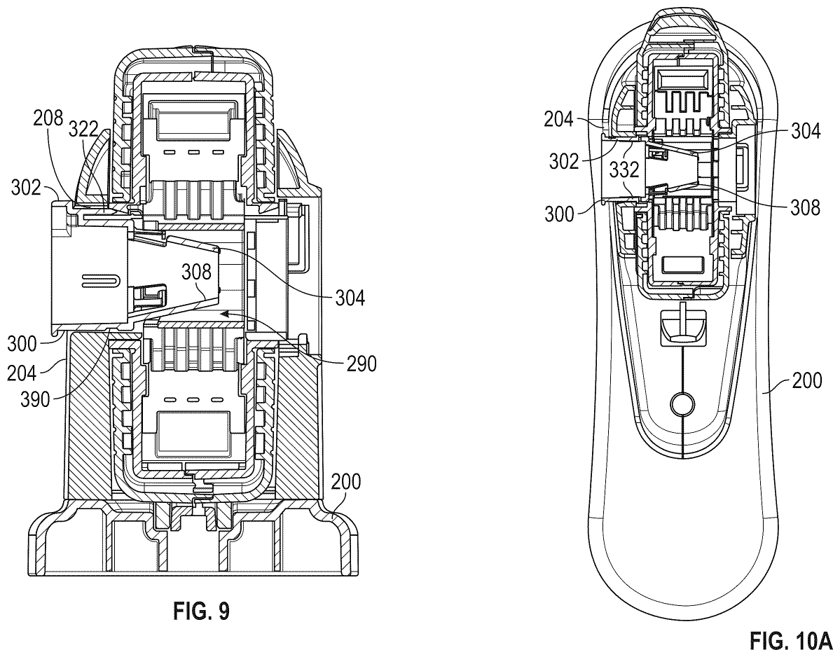

For example, as shown in FIG. 9, the alignment device 300 is inserted into the crimping device 200 such that the coupling member 322 slides into the cut-out portion 208.

The method at 404 can include, at 408, sliding the retaining member(s) 332 of the alignment device 300 along a surface 272 defining the channel 290 of the crimping device 200 as the alignment device 300 is inserted into the channel 290 and moving the retaining member(s) 332 radially outward and into the slot 270 in the crimping device 200 such that the alignment device 300 is retained in a specified axial orientation within the channel 290.

For example, as shown in FIGS. 10A and 10B, with the coupling member 322 engaged with the cut-out portion 208, the alignment device 300 slides axially into the channel 290 of the crimping device 200 until the retaining members 332 engage the slot 270 (or slots or recesses) in the crimping device 200.

More specifically, as the alignment device 300 moves axially into the channel 290, the protrusion 338 of each retaining member 332 slides along the surface 272 defining the channel 290 until it reaches the slot 270 and then the protrusion 338 snaps into the slot 270, thereby moving the retaining member 332 radially outward. As a result, the alignment device 300 is held within the channel 290 in a predetermined axial orientation relative to the crimping device (FIGS. 10A and 10B).

In some instances, as depicted in FIG. 10B, the surface 272 can have a ramped portion at the entrance to the channel 290. For example, the surface 272 can ramp radially inward from the distal face 204 of the crimping device toward an inside of the channel 290 such that the diameter of the channel 290 is slightly larger at its entrance (at the distal face 204) and then narrows to the main channel diameter. This provides a ramped surface 272 for the protrusions 338 of the retaining members 332 to slide along, thereby deflecting the retaining members 332 radially inward more gradually and increasing an ease of insertion of the alignment device 300 into the channel 290.

The method at 410 can include, prior to crimping, inserting a valve mounting portion of a delivery apparatus (e.g., delivery apparatus 100) into the channel 290, from the opposite side of the crimping device 200, and through the central channel of the alignment device. In some instances, a positioning device coupled to the delivery apparatus can be coupled to the proximal face of the crimping device 200, thereby holding the valve mounting portion of the delivery apparatus in a specified circumferential and axial orientation within the crimping device, and relative to the prosthetic valve.

The method at 412 can include radially compressing the prosthetic valve placed within the channel of the crimping device onto the valve mounting portion of the delivery apparatus by actuating the crimping device to move the pressing surfaces radially inwardly and, in response to actuating the crimping device and radially compressing the prosthetic valve, deflecting the alignment members of the alignment device radially inward and moving the alignment device axially out of and away from the prosthetic valve.

For example, during actuation of the crimping device 200 and crimping of the prosthetic valve to the valve mounting portion of the delivery apparatus, the pressing surfaces 284 of the crimping device 200 move radially inward toward the center of the channel 290, thereby radially compressing the prosthetic valve. As the pressing surfaces 284 move inward, they press on the alignment members 306 and cause the free ends 350 of the alignment members 306 to deflect radially inward and into the windows 352.

The pressing surfaces 284 can move further inward (in the radial direction), thereby pressing against the tapered support surface 310. The radially directed force of the pressing surfaces 284 against the tapered (or ramped) support surface 310 results in an axially directed force being applied to the alignment device 300 (in a distal direction, toward the distal face 204 of the crimping device 200, which is depicted by arrow 311 in FIG. 10B).

In response to this axially directed force, the protrusions 338 of the retaining members 332 are pushed out of the slot 270 in the crimping device 200, thereby deflecting the retaining members 332 radially inward. The protrusions 338 can slide along the surface 272 defining the channel 290 as the alignment device 300 is moved axially out of and away from the prosthetic valve, and out of the crimping device 200 (in the direction shown by arrow 311 in FIG. 10B).

As described above, the size (e.g., radius or height) of the protrusions 338 can be specified such that they are able to be released from the slot 270, in response to the axially directed force from the pressing surfaces 284 against the support surface 310. However, the retaining members 332 can be configured (e.g., due to their radially outward biassing relative to the surface 272 of the channel 290) such that the alignment device 300 does not suddenly eject out of the channel 290 during crimping.

In some examples, the alignment device 300 can remain at least partially inside the channel 290 following crimping (e.g., when the pressing surfaces 284 have finished radially compressing the valve onto the delivery apparatus). In some examples, the alignment device 300 can move out of and/or fall out of the crimping device 200 slowly as the pressing surfaces 284 radially compress the valve onto the delivery apparatus.

Further details on crimping a prosthetic valve mounted on support body to the valve mounting portion of the delivery apparatus and coupling a positioning device coupled to a delivery apparatus to the crimping device can be found in in PCT Patent Publication Nos. WO 2022/046585 and WO 2022/046319, which are incorporated by reference herein in their entireties.

By utilizing the alignment device 300 (or a similar alignment device) for rotationally and axially aligning a prosthetic valve within a crimping device, relative to a delivery apparatus arranged within the crimping device, the prosthetic valve can be radially compressed onto a valve mounting portion of the delivery apparatus in a specified circumferential orientation and axial orientation relative to the delivery apparatus.

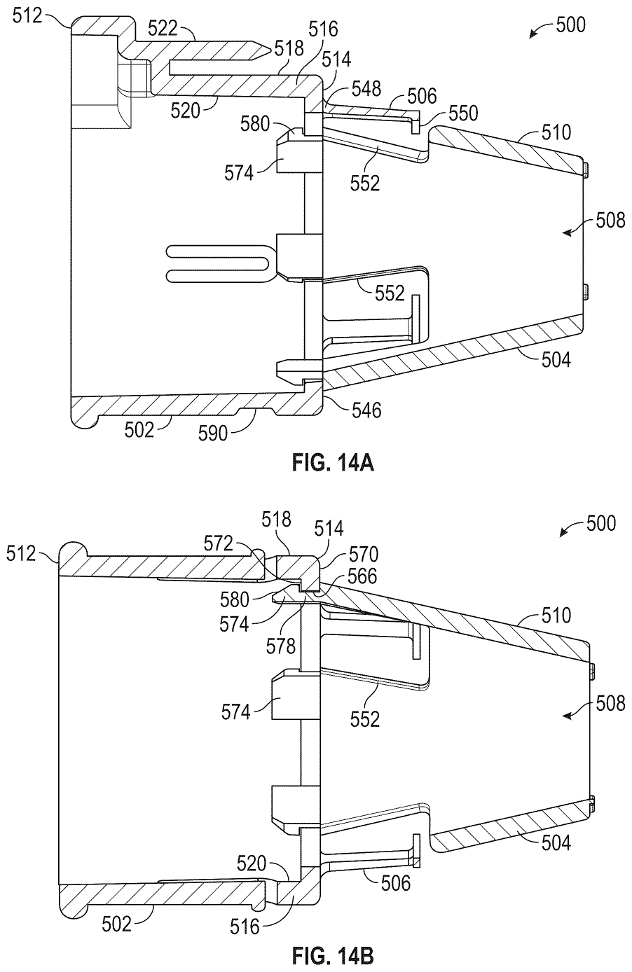

FIGS. 12-16 depict an exemplary alignment device 500 that is configured to receive a prosthetic valve thereon and couple to a crimping device. As shown in the exploded perspective view of FIG. 12, the end view of FIG. 13, and the cross-sectional views of FIGS. 14A and 14B, the alignment device 500 includes a coupling portion 502 and a support portion 504 (valve support portion). The alignment device 500 also includes one or more alignment members 506 which are part of (formed or molded together as one piece or unitary component with) the coupling portion 502.

The alignment device 500 can be the same or similar to the alignment device 300 of FIGS. 4-5B, as described above, except the coupling portion 502 and the support portion 504 are separate parts that can be coupled together for use (as described above with reference to FIG. 11). In some examples, the coupling portion 502 and the support portion 504 are removably coupled to (and separable from) one another. As such, the alignment device 500 is a two-part alignment device comprising the coupling portion 502 and the support portion 504, and the alignment members 506 are part of the coupling portion 502. Thus, the coupling portion 502 can also be referred to as a coupling component 502 and the support portion 504 can also be referred to as a support component 504.

The components of the alignment device 500 that correspond to (are the same or similar to) the components of the alignment device 300 are labeled similarly in FIGS. 12-16. For example, alignment members 506 of alignment device 500 correspond to and are the same as the alignment members 306 of alignment device 300 and are not redescribed below for the sake of brevity. In some examples, coupling member 522 of alignment device 500 is the same as the coupling member 322 of alignment device 300.

The alignment device 500 also includes a central channel 508 extending through the coupling portion 502 and the support portion 504 when they are coupled together (as shown in FIGS. 14A and 14B). The central channel 508 is configured to receive a delivery apparatus for the prosthetic valve therethrough (e.g., delivery apparatus 100).

The same as described above for the alignment device 300, the coupling portion 502 can be configured to releasably couple to a crimping device, such as the crimping device 200 shown in FIGS. 3A-3B and 7-10B. The support portion 504 can be configured to be inserted into a prosthetic valve (for example, prosthetic valve 50) so that leaflets (for example, leaflets 60) of the prosthetic valve can contact and rest upon the support surface 510 of the support portion 504. The alignment members 506 can be used to space the prosthetic valve away from the cylindrical wall 516 of the coupling portion 502 and achieve a desired rotational alignment of the prosthetic valve relative to the coupling portion 502 and the crimping device.

The coupling portion 502 (or coupling component) comprises a cylindrical wall 516, which has an outer surface 518 and an inner surface 520 (FIGS. 12-14B and 16). The inner surface 520 defines a lumen of the coupling portion 502 that forms a portion of the central channel 508. The cylindrical wall 516 has a first end 512 (distal end) and a second end 514 (proximal end).

The coupling portion 502 can include a coupling member 522 that is configured to rotationally align the coupling portion 502 with a crimping device, the same as described above for the coupling portion 302 of the alignment device 300. For example, the coupling member 522 can be the same as coupling member 322, and thus can be configured (shaped) to mate with the cut-out portion 208 (or recess 208) of the crimping device 200.

In some examples, the coupling member 522 can extend or protrude outward from a base element 530 of the coupling portion 502 (where the base element 530 is the same or similar to base element 330).

The cylindrical wall 516 of the coupling portion 502 can include one or more retaining members 532 (the same as retaining members 332) which are configured as axial locking mechanisms that prevent the coupling portion 502 from moving axially relative to the crimping device 200 after the alignment device 500 is attached to the crimping device 200 (the same as described above for the coupling portion 302 of the alignment device 300).

The coupling portion 502 can mate with the crimping device, the same as shown in FIGS. 7-10B, as described above.

In some examples, the cylindrical wall 516 can include a flat indentation 590 that is depressed into the outer surface 518 toward the inner surface 520 (FIG. 14A).

The axially extending alignment members 506 (which can be the same as alignment members 306, as described above) are formed as part of the coupling portion 502 (e.g., formed or molded together as one piece or a single unitary component). For example, in some instances, the coupling portion 502, including the alignment members 506, can be injection molded with a single mold.

As shown in FIGS. 12, 14A-14B, and 16, each alignment member 506 includes an attached end 548 disposed at the axial wall 546 and a free end 550 disposed axially away from the attached end 548. Each alignment member 506 extends axially outward from the axial wall 546, over a portion of the support portion 504 and to the support surface 510 when the coupling portion 502 and support portion 504 are coupled together (FIGS. 14A-14B).

It should be noted that when the coupling portion 502 and support portion 504 are coupled or assembled together, the alignment device 500 can appear identical to the alignment device 300 shown in FIG. 4.

The axial wall 546 comprises indentations 566 (or indented regions) that are spaced apart from one another around a circumference of the radially inward facing surface 568 of the axial wall 546 (FIGS. 12, 14B, and 16).

The axial wall 546 can have an axially outward facing surface 570 and an axially inward facing surface 572 (FIG. 14B), and the radially inward facing surface 568 extends therebetween.

In some examples, the axial wall 546 comprises three circumferentially extending and spaced apart indentations 566 in the radially inward facing surface 568.

The support portion 504 is configured similar to the support portion 304 of the alignment device 300, except the support portion 504 includes tabs 574 (FIGS. 12-15). The tabs 574 are configured to couple to the indentations 566 in the axial wall 546 of the coupling portion 502, thereby coupling the support portion 504 to the coupling portion 502.

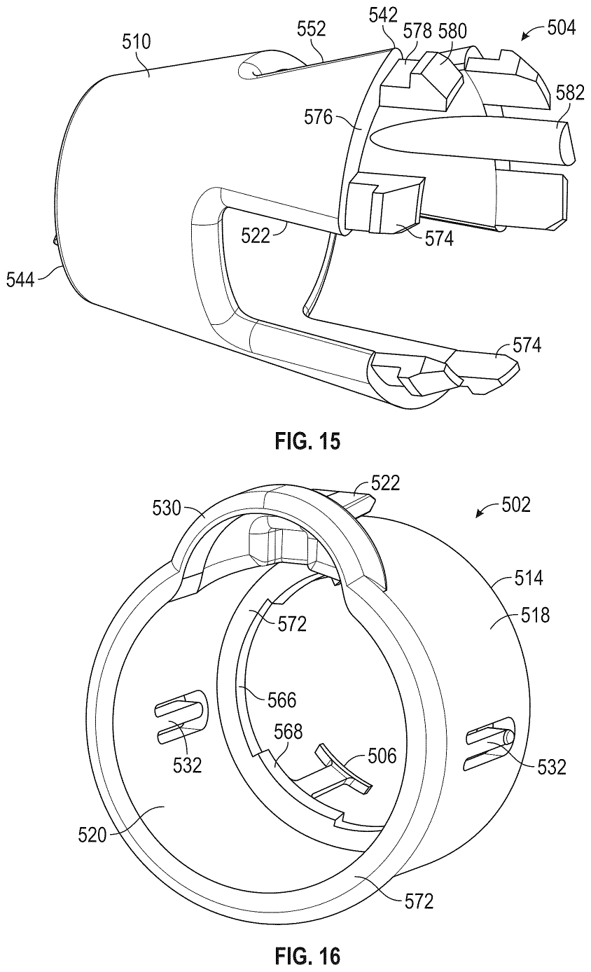

For example, a plurality of tabs 574 extend axially outward (e.g., in the distal direction) from an axial wall 576 that is disposed at the first end 542 (distal end) of the support portion 504 (FIG. 15). In some examples, the tabs 574 can be grouped together in pairs of tabs 574 that are spaced apart from one another.

For example, the axial wall 576 can be broken into circumferentially spaced apart sections by the windows 552. In some examples, two tabs 574 (or one pair of tabs) can extend axially outward from each section of the axial wall 576, wherein the two tabs 574 are spaced apart from each other in the section of the axial wall 576 (FIGS. 12 and 15).

In some examples, each section of the axial wall 576 can include more or less than two tabs 574 (e.g., one, three, or the like).

In some examples, instead of two or more smaller tabs 574, each section of the axial wall 576 can include one larger tab (larger width in the circumferential direction) extending axially therefrom.

Each tab 574 can comprise a shaft 578 with a tapered tip 580 at its free end (FIGS. 14B and 15). The shaft 578 is configured to fit (or interface) with a corresponding indentation 566 and the tapered tip 580 is configured to couple with the axially inward facing surface 572 of the axial wall 546 of the coupling portion 502. For example, the tapered tip 580 can be configured as a hook that hooks around the axial wall 546 (at the indentation 566).

In some examples, each tab 574 can be configured as a cantilever beam that can flex such that the tapered tip 580 snaps into engagement with the indentation 566 (as shown in the assembled views of FIGS. 13-14B).

In some examples, the support portion 504 can include an axially extending member 582 (FIGS. 12, 13, and 15) disposed between (and spaced apart from) a pair of tabs 574. For example, as shown in FIG. 15, the axially extending member 582 can extend axially outward from the axial wall 576, between two tabs 574. The axially extending member 582 can be a remnant of the molding (manufacturing) process and can allow for an injection molded sub-gate to fill the mold to make the alignment device 500.

In some examples, the alignment device 500 may not include the axially extending member 582 (e.g., it may be removed after the molding process).

The support portion 504 is configured to rotationally align the prosthetic valve on a support surface 510 of the support portion 504, be inserted into the channel 290 of the crimping device 200 and receive a delivery apparatus through the central channel 508. The support portion 504 can be configured and function the same as the support portion 304 of the alignment device 300 (except for the inclusion of the tabs 574, as described above). As such, the support portion 504 tapers from the first end 542 to its second end 544. As a result, the support surface 510 which is configured to receive the prosthetic heart valve thereon, also tapers in diameter.

The support portion 504 and the coupling portion 502 can be manufactured separately and comprise different materials.

For example, the support portion 504 can be a first material. In some examples, the first material is a higher durometer material, such as polyetheretherketone (PEEK). The higher durometer material of the support portion 504 can enable the support portion 504 to support the prosthetic valve thereon and withstand forces from the pressing surfaces 284 of the crimping device 200, as the prosthetic valve is radially compressed by the pressing surfaces 284.

The coupling portion 502 can be a second material. In some examples, the second material can be a lower durometer material, such as polycarbonate, polyethylene terephthalate (PET), acrylonitrile butadiene styrene (ABS), or the like, or combinations thereof.

As a result of the coupling portion 502 being constructed of a lower durometer material, the alignment members 506 can more easily flex due to a lower flexural modulus of the lower durometer material (the second material).

In some examples, the lower durometer, second material may be lower cost (as compared to the higher durometer material, e.g., PEEK), thereby reducing costs for manufacturing the coupling portion 502, and thus reducing overall manufacturing costs of the alignment device 500 (as compared to an alignment device which is entirely composed of PEEK or another higher durometer material).

Additionally, the individual molds for the coupling portion 502 and the support portion 504 may be easier to manufacture (as compared to forming a single mold for an integrated or one-piece alignment device, which may be more complicated in some instances).

Further, the coupling portion 502 may be easier to mold (or form within the mold), due to better flow with the lower durometer material.

In some examples, the lower durometer (or softer) material of the coupling portion 502 may be easier to dye (or color), as compared to the higher durometer material. For example, in some instances, the coupling portion 502 can be various colors, and the color of the coupling portion 502 can be specified such that it corresponds to a specific size prosthetic valve to be used with the alignment device 500.

For example, a first alignment device 500 can be configured (sized) to be used with a first sized prosthetic valve, and thus the coupling portion 502 of the first alignment device 500 can have a first color that corresponds to the first sized alignment device 500 and prosthetic valve. A second alignment device 500 can be configured (sized) to be used with a second sized prosthetic valve, and thus the coupling portion 502 of the first alignment device 500 can have a second color that corresponds to the second sized alignment device 500 and prosthetic valve. In this way, the material of the coupling portion 502 can be color coded for alignment device and valve size identification.

Although the examples described above with reference to FIGS. 4-16 are directed to prosthetic heart valves, the disclosed devices and methods can be used with other implantable medical devices including stents, grafts, and the like (e.g., in order to crimp and mount the implantable medical device onto a delivery apparatus).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Additionally and/or alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Additionally and/or alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Additionally and/or alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An alignment device for a prosthetic heart valve, comprising: a coupling portion comprising a coupling member configured to couple with a crimping device; a support portion that tapers from a wider end that is disposed adjacent a first end of the coupling portion to a narrower end, wherein the support portion comprises an outward facing support surface that is configured to receive the prosthetic heart valve thereon; one or more axially extending alignment members, each alignment member extending axially outward from the first end of the coupling portion, over the support portion and to the support surface, each alignment member comprising a free end configured to abut an end of the prosthetic heart valve and be axially aligned with a commissure of the prosthetic heart valve when the prosthetic heart valve is arranged on the support surface; and a central channel extending through the coupling portion and the support portion, the central channel configured to receive a delivery apparatus for the prosthetic heart valve therethrough.

Example 2. The alignment device of any example herein, particularly example 1, wherein the free end of each alignment member is configured to deflect radially inward toward the central channel.

Example 3. The alignment device of any example herein, particularly either example 1 or example 2, wherein the support portion includes one or more windows, each window extending from the wider end to the support surface.

Example 4. The alignment device of any example herein, particularly example 3, wherein each window extends through a wall of the support portion, from an outer surface to an inner surface of the support portion.

Example 5. The alignment device of any example herein, particularly either example 3 or example 4, wherein each window has a width, in a circumferential direction, that is wider than the free end of a corresponding alignment member.

Example 6. The alignment device of any example herein, particularly any one of examples 1-5, wherein each alignment member is T-shaped, wherein a wider portion of the T shape forms the free end of each alignment member, and wherein a shaft of the T shape is attached to the first end of the coupling portion.

Example 7. The alignment device of any example herein, particularly example 6, wherein the free end of each alignment member curves in a circumferential direction, around a central longitudinal axis of the alignment device.

Example 8. The alignment device of any example herein, particularly either example 6 or example 7, wherein an axial face of the free end of each alignment member is planar, and wherein the axial face is normal to a central longitudinal axis of the alignment device.

Example 9. The alignment device of any example herein, particularly any one of examples 1-8, wherein the one or more axially extending alignment members includes three alignment members spaced circumferentially apart from one another around the alignment device.

Example 10. The alignment device of any example herein, particularly any one of examples 1-9, wherein the coupling portion comprises a cylindrical wall with an outer surface and an inner surface at least partially defining the central channel, and wherein the cylindrical wall extends from a first end to an opposite, second end of the cylindrical wall.

Example 11. The alignment device of any example herein, particularly example 10, wherein the coupling member extends axially from the second end toward the first end, and wherein the coupling member is spaced radially away from the outer surface of the cylindrical wall.

Example 12. The alignment device of any example herein, particularly example 11, wherein the coupling member has a free end that tapers from its opposing radially outward facing and radially inward facing surfaces to a point.

Example 13. The alignment device of any example herein, particularly example 10, further comprising at least one retaining member disposed in the cylindrical wall of the coupling portion, wherein the retaining member is configured to retain an axial position of the alignment device inside a channel of the crimping device.

Example 14. The alignment device of any example herein, particularly example 13, wherein the at least one retaining member comprises a finger with a free end and attached end that are formed by a U-shaped slot in the cylindrical wall, wherein the free end includes a protrusion that protrudes radially outward from an outer surface of the free end, and wherein the free end is configured to deflect radially inward and outward relative to the attached end.

Example 15. The alignment device of any example herein, particularly example 14, wherein the protrusion is a spherical nub.

Example 16. The alignment device of any example herein, particularly any one of examples 13-15, wherein the at least one retaining member includes two retaining members disposed in opposite sides of the cylindrical wall of the coupling portion.

Example 17. The alignment device of any example herein, particularly any one of examples 1-16, wherein the support portion comprises one or more alignment indicators disposed at the narrower end, and wherein each alignment indicator is axially aligned with a corresponding alignment member of the one or more alignment members.

Example 18. The alignment device of any example herein, particularly any one of examples 1-17, wherein the support surface is a conical surface that is configured to hold one or more leaflets of the prosthetic heart valve in an open position.

Example 19. The alignment device of any example herein, particularly any one of examples 1-18, wherein the coupling portion and the support portion are separate parts that are configured to be coupled to one another, and wherein the one or more alignment members are molded together with the coupling portion.

Example 20. The alignment device of any example herein, particularly example 19, wherein the coupling portion, including the one or more alignment members, comprises a first material, and wherein the support portion comprises a second material.

Example 21. The alignment device of any example herein, particularly example 20, wherein the second material has a higher durometer than the first material.

Example 22. The alignment device of any example herein, particularly either example 20 or example 21, wherein the first material is one of polycarbonate, PET, and ABS.

Example 23. The alignment device of any example herein, particularly any one of examples 20-22, wherein the second material is PEEK.

Example 24. The alignment device of any example herein, particularly any one of examples 19-23, wherein the support portion comprises a plurality of tabs extending axially away from the wider end of the support portion, toward the coupling portion, which are configured to couple to the first end of the coupling portion.

Example 25. The alignment device of any example herein, particularly example 24, wherein the first end of the coupling portion comprises an axial wall and a plurality of indentations in a radially inward facing surface of the axial wall, and wherein one or more tabs of the plurality of tabs are configured to couple with a respective indentation of the plurality of indentations.

Example 26. The alignment device of any example herein, particularly either example 24 or example 25, wherein each tab is configured as a cantilever beam that snap fits with the first end of the coupling portion such that the support portion and coupling portion are coupled together.

Example 27. An assembly comprising the alignment device of any example herein, particularly any one of examples 1-26, and the prosthetic heart valve.

Example 28. A system for crimping a prosthetic heart valve to a delivery apparatus, comprising: the alignment device of any one of claims 1-26; and the crimping device which comprises a channel, wherein a first opening to the channel is disposed on a distal side of the crimping device, the first opening configured to receive the support portion of the alignment device therethrough, and wherein the crimping device includes a recess in the distal side that is configured to receive the coupling member of the alignment device therein.

Example 29. The system of any example herein, particularly example 28, wherein the channel of the crimping device includes a slot, and wherein the coupling portion of the alignment device further comprises a retaining member that is configured to couple with the slot such that the alignment device is held in a specified axial orientation within and relative to the crimping device.

Example 30. The system of any example herein, particularly example 29, wherein the retaining member is a first retaining member, and wherein the coupling portion comprises a second retaining member spaced circumferentially apart from the first retaining member.

Example 31. The system of any example herein, particularly example 29, wherein the retaining member is disposed in a cylindrical wall of the coupling portion, the cylindrical wall having an outer surface and an opposite, inner surface that at least partially defines the central channel of the alignment device.

Example 32. The system of any example herein, particularly example 31, wherein the retaining member comprises a finger with a free end and attached end that are formed by a U-shaped slot in the cylindrical wall, wherein the free end includes a protrusion that protrudes radially outward from an outer surface of the free end, and wherein the free end is configured to deflect radially inward and outward relative to the attached end.

Example 33. The system of any example herein, particularly example 32, wherein the protrusion is a spherical nub.

Example 34. The system of any example herein, particularly either example 32 or example 33, wherein a surface that defines the channel of the crimping device has a ramped portion at an entrance to the channel, wherein the ramped portion ramps radially inward from the distal side of the crimping device toward an inside of the channel, and wherein the protrusion is configured to slide along the surface to the slot.

Example 35. The system of any example herein, particularly any one of examples 28-34, wherein the coupling member extends axially outward from a second end of the coupling portion toward the first end of the coupling portion, and wherein the coupling member is shaped to fit within the recess of the crimping device and circumferentially align the alignment device within and relative to the crimping device.

Example 36. A method comprising: placing a prosthetic valve in a radially expanded configuration over a support surface of a tapered support portion of an alignment device and rotationally aligning the prosthetic valve on the support surface such that commissures of the prosthetic valve are axially aligned with alignment members of the alignment device, wherein the support portion extends from a proximal end of a coupling portion of the alignment device, wherein the alignment members extend axially outward from the proximal end of the coupling portion, over the support portion, and to the support surface, and wherein the coupling portion includes a coupling member that extends axially outward from a distal end of the coupling portion toward the support portion; inserting the prosthetic valve placed over the support surface into a channel of a crimping device, the channel being surrounded by a plurality of pressing surfaces of the crimping device; inserting the coupling member into a complementary recess in a distal face of the crimping device such that the alignment device is held in a specified circumferential orientation relative to the crimping device; and radially compressing the prosthetic valve placed within the channel by actuating the crimping device to move the pressing surfaces radially inwardly, wherein the radially compressing causes the alignment members to deflect radially inward and the alignment device to be moved axially out of and away from the prosthetic valve.

Example 37. The method of any example herein, particularly example 36, further comprising, prior to the radially compressing the prosthetic valve by actuating the crimping device, inserting a valve mounting portion of a delivery apparatus into the channel of the crimping device and through a central channel of the alignment device, and wherein the radially compressing the prosthetic valve includes radially compressing the prosthetic valve onto the valve mounting portion of the delivery apparatus.

Example 38. The method of any example herein, particularly either example 36 or example 37, wherein the coupling portion of the alignment device comprises a cylindrical wall and a retaining member disposed in the cylindrical wall, and further comprising sliding the retaining member along a surface defining the channel of the crimping device as the alignment device is inserted into the channel and moving the retaining member radially outward and into a slot in the crimping device that is radially offset from the channel such that the alignment device is retained in a specified axial orientation within the channel.

Example 39. The method of any example herein, particularly example 38, wherein the radially compressing further causes the alignment member to uncouple from the slot and allow the alignment device to be moved axially out of and away from the prosthetic valve.

Example 40. The method of any example herein, particularly either example 38 or example 39, wherein the surface defining the channel of the crimping device comprises a ramped portion at an entrance to the channel which ramps radially inward from the distal face of the crimping device toward an inside of the channel, wherein the retaining member is configured as a cantilever with a free end that is deflectable in a radial direction and that comprises a protrusion, and wherein sliding the retaining member along the surface defining the channel includes sliding the protrusion along the surface such that the free end of the retaining member deflects radially inward until it reaches the slot and then deflecting the free end of the retaining member radially outward such that the protrusion is disposed within the slot.

Example 41. The method of any example herein, particularly any one of examples 36-40, wherein placing the prosthetic valve over the support surface and rotationally aligning the prosthetic valve on the support surface includes abutting an end of the prosthetic valve against free ends of the alignment members, and wherein the free ends of the alignment members are wider than attached ends of the alignment members that are attached to the proximal end of the coupling portion.

Example 42. The method of any example herein, particularly any one of examples 36-41, wherein the radially compressing causes the free ends of the alignment members to deflect radially inward into respective windows disposed in the support portion, adjacent to the support surface.

Example 43. The method of any example herein, particularly any one of examples 36-42, wherein the alignment device comprises three alignment members, and wherein rotationally aligning the prosthetic valve on the support surface includes axially aligning each commissure of three commissures of the prosthetic valve with a corresponding alignment member of the three alignment members of the alignment device.

Example 44. The method of any example herein, particularly any one of examples 36-43, wherein the coupling portion and the support portion are removably coupled to one another, and wherein the one or more alignment members are formed as one piece with the coupling portion.

Example 45. The method of any example herein, particularly example 44, wherein the coupling portion and the support portion are snap fit together.

Example 46. The method of any example herein, particularly any one of examples 36-45, wherein the coupling portion and alignment members are molded together as a unitary component that comprises a first material, and wherein the support portion is molded as a separate component and comprises a second material.

Example 47. The method of any example herein, particularly example 46, wherein the second material has a higher durometer than the first material.

Example 48. An alignment device for a prosthetic heart valve, comprising: a coupling portion comprising a cylindrical wall with a first end and a second end, and a retaining member disposed in the cylindrical wall, wherein the retaining member is configured to retain an axial position of the alignment device inside a channel of a crimping device; a support portion that tapers from a wider end disposed at the second end of the coupling portion to a narrower end, wherein the support portion comprises an outward facing support surface that is configured to receive the prosthetic heart valve thereon; an axially extending alignment member, the alignment member extending axially outward from the second end of the cylindrical wall of the coupling portion, over the support portion and to the support surface, wherein the alignment member is configured to abut an end of the prosthetic heart valve and be axially aligned with a specified feature of the prosthetic heart valve when the prosthetic heart valve is arranged on the support surface; and a central channel extending through the alignment device, the central channel configured to receive a delivery apparatus for the prosthetic heart valve therethrough.

Example 49. The alignment device of any example herein, particularly example 48, wherein the coupling portion comprises a coupling member configured to couple with the crimping device and retain the alignment member in a specified circumferential orientation within the channel of the crimping device.

Example 50. The alignment device of any example herein, particularly example 49, wherein the coupling member extends axially outward from the first end of the cylindrical wall of the coupling portion toward the second end, and wherein the coupling member is spaced radially away from an outer surface of the cylindrical wall.

Example 51. The alignment device of any example herein, particularly example 50, wherein the coupling member has a free end that tapers from opposite sides of the coupling member to a tapered end.

Example 52. The alignment device of any example herein, particularly any one of examples 48-51, wherein the alignment member is radially offset from the support member, wherein the alignment member comprises an attached end disposed at the second end of the cylindrical wall of the coupling portion and a free end that is configured to deflect in a radial direction relative to the attached end, and wherein the free end is configured to abut an end of the prosthetic heart valve when the prosthetic heart valve is arranged on the support surface.

Example 53. The alignment device of any example herein, particularly example 52, wherein the support portion includes a window that extends from the wider end to the support surface and is configured to receive the free end of the alignment member therein.

Example 54. The alignment device of any example herein, particularly example 53, wherein the window extends through a wall of the support portion, from an outer surface to an inner surface of the support portion, and wherein the inner surface at least partially defines the central channel.

Example 55. The alignment device of any example herein, particularly any one of examples 52-54, wherein the alignment member is T-shaped, wherein a wider portion of the T shape forms the free end, and wherein a shaft of the T shape is attached to the second end of the cylindrical wall of the coupling portion.

Example 56. The alignment device of any example herein, particularly example 55, wherein the free end curves in a circumferential direction, around a central longitudinal axis of the alignment device.

Example 57. The alignment device of any example herein, particularly either example 55 or example 56, wherein an axial face of the free end is planar, and wherein the axial face is normal to a central longitudinal axis of the alignment device.

Example 58. The alignment device of any example herein, particularly any one of examples 48-57, wherein the alignment member is a first alignment member, and further comprising two additional alignment members that are spaced circumferentially apart from each other and the first alignment member.

Example 59. The alignment device of any example herein, particularly any one of examples 48-58, wherein the retaining member comprises a finger with a free end and attached end that are formed by a U-shaped slot in the cylindrical wall, wherein the free end includes a protrusion that protrudes radially outward from an outer surface of the free end, and wherein the free end is configured to deflect radially inward and outward relative to the attached end.

Example 60. The alignment device of any example herein, particularly example 59, wherein the protrusion is a spherical nub.

Example 61. The alignment device of any example herein, particularly either example 59 or example 60, wherein the retaining member is a first retaining member, wherein the coupling portion comprises a second retaining member disposed in the cylindrical wall, and wherein the first and second retaining members are spaced circumferentially apart from one another.

Example 62. The alignment device of any example herein, particularly any one of examples 48-61, wherein the specified feature is a commissure of the prosthetic heart valve.

Example 63. The alignment device of any example herein, particularly any one of examples 48-61, wherein the coupling portion, support portion, and axially extending alignment member are formed together as a unitary component.

Example 64. The alignment device of any example herein, particularly any one of examples 48-62, wherein the coupling portion and the support portion are separate parts that are configured to be coupled to one another, and wherein the one or more alignment members are molded together with the coupling portion to form a unitary component.

Example 65. The alignment device of any example herein, particularly example 64, wherein the coupling portion, including the one or more alignment members, comprises a first material, and wherein the support portion comprises a second material.

Example 66. The alignment device of any example herein, particularly example 65, wherein the first material has a first durometer and the second material has a second durometer, and wherein the second durometer is larger than the first durometer.

Example 67. The alignment device of any example herein, particularly either example 65 or example 66, wherein the first material is one of polycarbonate, PET, and ABS.

Example 68. The alignment device of any example herein, particularly any one of examples 65-67, wherein the second material is PEEK.

Example 69. The alignment device of any example herein, particularly any one of examples 64-68, wherein the support portion comprises a plurality of circumferentially spaced apart tabs extending axially away from the wider end of the support portion, toward the coupling portion, which are configured to couple to the second end of the coupling portion such that the wider end of the support portion abuts the second end of the coupling portion and the coupling portion and support portion are coupled together.

Example 70. The alignment device of example herein, particularly example 69, wherein the second end of the coupling portion comprises an axial wall and a plurality of circumferentially spaced apart indentations in a radially inward facing surface of the axial wall, and wherein one or more tabs of the plurality of tabs are configured to couple with a respective indentation of the plurality of indentations.

Example 71. The alignment device of any example herein, particularly either example 69 or example 70, wherein each tab is configured as a cantilever beam that snap fits with the second end of the coupling portion such that the support portion and coupling portion are coupled together.

Example 72. An alignment device for a prosthetic heart valve, comprising: a coupling portion comprising: a cylindrical wall with a first end and a second end, wherein the second end comprises an axial wall, and wherein a plurality of indentations is disposed in a radially inward facing surface of the axial wall; a coupling member extending axially from the first end toward the second end of the cylindrical wall, wherein the coupling member is configured to couple with a crimping device; and one or more axially extending alignment members, each alignment member extending axially outward from the second end of the cylindrical wall; a support portion that tapers from a wider, first end to a narrower, second end, wherein the support portion comprises: an outward facing support surface that is configured to receive the prosthetic heart valve thereon, wherein the one or more axially extending alignment members extend over the support portion and to the support surface, each alignment member comprising a free end configured to abut an end of the prosthetic heart valve and be axially aligned with a commissure of the prosthetic heart valve when the prosthetic heart valve is arranged on the support surface; and a plurality of tabs extending axially away from the first end of the support portion, wherein the plurality of tabs is configured to couple to the plurality of indentations such that the coupling portion and support portion are coupled to one another; and a central channel extending through the coupling portion and the support portion, the central channel configured to receive a delivery apparatus for the prosthetic heart valve therethrough.

Example 73. The alignment device of any example herein, particularly example 72, wherein the coupling portion and the support portion are separately molded components and removable from one another.

Example 74. The alignment device of any example herein, particularly either example 72 or example 73, wherein the coupling portion comprises a first material, and wherein the support portion comprises a second material.

Example 75. The alignment device of any example herein, particularly example 74, wherein the second material has a higher durometer than the first material.

Example 76. The alignment device of any example herein, particularly either example 74 or example 75, wherein the first material is one of polycarbonate, PET, and ABS.

Example 77. The alignment device of any example herein, particularly any one of examples 74-76, wherein the second material is PEEK.

Example 78. The alignment device of any example herein, particularly any one of examples 72-77, wherein each tab of the plurality of tabs is configured as a cantilever beam that snap fits with a corresponding indentation of the plurality of indentations such that the support portion and coupling portion are coupled together.

Example 79. The alignment device of any example herein, particularly any one of examples 72-78, wherein the plurality of tabs is grouped into a plurality of pairs of tabs that are circumferentially spaced apart around the cylindrical wall, and wherein each pair of tabs is configured to couple with a corresponding indentation of the plurality of indentations.

Example 80. The alignment device of any example herein, particularly any one of examples 72-79, wherein the coupling member extends axially from the first end toward the second end, and wherein the coupling member is spaced radially away from an outer surface of the cylindrical wall.

Example 81. The alignment device of any example herein, particularly any one of examples 72-80, wherein the coupling portion further comprises at least one retaining member disposed in the cylindrical wall, and wherein the retaining member is configured to retain an axial position of the alignment device inside a channel of the crimping device.

Example 82. The alignment device of any example herein, particularly example 81, wherein the at least one retaining member is configured as a cantilever with a free end and attached end that are formed by a U-shaped slot in the cylindrical wall, wherein the free end includes a protrusion that protrudes radially outward from an outer surface of the free end, and wherein the free end is configured to deflect radially inward and outward relative to the attached end.

Example 83. The alignment device of any example herein, particularly either example 81 or example 82, wherein the at least one retaining member includes two retaining members disposed in opposite sides of the cylindrical wall of the coupling portion.

Example 84. The alignment device of any example herein, particularly any one of examples 72-83, wherein the free end of each alignment member is configured to deflect radially inward toward the central channel.

Example 85. The alignment device of any example herein, particularly any one of examples 72-84, wherein the support portion includes one or more windows, each window extending from the wider end to the support surface, and wherein each window is configured to receive the free end of a corresponding alignment member.

Example 86. The alignment device of any example herein, particularly example 85, wherein each window extends through a wall of the support portion, from an outer surface to an inner surface of the support portion.

Example 87. The alignment device of any example herein, particularly any one of examples 72-86, wherein each alignment member is T-shaped, wherein a wider portion of the T shape forms the free end of each alignment member, and wherein a shaft of the T shape is attached to the axial wall at the second end of the cylindrical wall.

Example 88. The alignment device of any example herein, particularly any one of examples 72-87, wherein the one or more axially extending alignment members includes three alignment members spaced circumferentially apart from one another around the coupling portion.

Example 89. An alignment device for a prosthetic heart valve, comprising: a coupling portion comprising a cylindrical wall with a first end and a second end, and a coupling member configured to couple with a crimping device, wherein the coupling member extends axially outward from the first end toward the second; a support portion that tapers radially inward from a wider end that is disposed adjacent the second end of the coupling portion to a narrower end, wherein the support portion comprises: an outward facing support surface that is configured to receive the prosthetic heart valve thereon; and a plurality of alignment indicators spaced circumferentially apart from one another at the narrower end of the support portion, wherein each alignment indicator is configured to be axially aligned with a commissure of the prosthetic heart valve when the prosthetic heart valve is arranged on the support surface; and a central channel extending through the coupling portion and the support portion, the central channel configured to receive a delivery apparatus for the prosthetic heart valve therethrough.

Example 90. The alignment device of any example herein, particularly example 89, wherein the plurality of alignment indicators are arrows.

Example 91. The alignment device of any example herein, particularly example 89 or 90, wherein the narrower end of the support portion comprises an axial wall, and wherein the plurality of alignment indicators is disposed in the axial wall.

Example 92. The alignment device of any example herein, particularly any one of examples 89-91, further comprising a plurality of axially extending alignment members, each alignment member extending axially outward from the second end of the coupling portion, over the support portion and to the support surface, each alignment member comprising a free end configured to abut an end of the prosthetic heart valve and be axially aligned with a commissure of the prosthetic heart valve when the prosthetic heart valve is arranged on the support surface, and wherein each alignment member is axially aligned with a corresponding alignment indicator of the plurality of alignment indicators.

Example 93. A method comprising sterilizing the prosthetic heart valve, alignment device, system, and/or assembly of any example.

Example 94. A prosthetic heart valve of any one of examples 1-88, wherein the prosthetic heart valve is sterilized.

The features described herein with regard to any example can be combined with other features described in any one or more of the other examples, unless otherwise stated. For example, any one or more of the features of one alignment device can be combined with any one or more features of another alignment device. As another examples, any one or more features of one delivery apparatus can be combined with any one or more features of another delivery apparatus. As another example, any one or more features of one crimping device can be combined with any one or more features of another crimping device.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

What is claimed is:

1. An alignment device for a prosthetic heart valve, comprising:
a coupling portion comprising a coupling member configured to couple with a crimping device;
a support portion that tapers from a wider end that is disposed adjacent a first end of the coupling portion to a narrower end, wherein the support portion comprises an outward facing support surface that is configured to receive the prosthetic heart valve thereon;
one or more axially extending alignment members, each alignment member extending axially outward from the first end of the coupling portion, over the support portion and to the support surface, each alignment member comprising a free end configured to abut an end of the prosthetic heart valve and be axially aligned with a commissure of the prosthetic heart valve when the prosthetic heart valve is arranged on the support surface; and
a central channel extending through the coupling portion and the support portion, the central channel configured to receive a delivery apparatus for the prosthetic heart valve therethrough.

2. The alignment device of claim 1, wherein the free end of each alignment member is configured to deflect radially inward toward the central channel.

3. The alignment device of claim 1, wherein the support portion includes one or more windows, each window extending from the wider end to the support surface.

4. The alignment device of claim 3, wherein each window has a width, in a circumferential direction, that is wider than the free end of a corresponding alignment member.

5. The alignment device of claim 1, wherein each alignment member is T-shaped, wherein a wider portion of the T shape forms the free end of each alignment member, and wherein a shaft of the T shape is attached to the first end of the coupling portion.

6. The alignment device of claim 1, wherein the coupling portion comprises a cylindrical wall with an outer surface and an inner surface at least partially defining the central channel, and wherein the cylindrical wall extends from a first end to an opposite, second end of the cylindrical wall.

7. The alignment device of claim 6, further comprising at least one retaining member disposed in the cylindrical wall of the coupling portion, wherein the retaining member is configured to retain an axial position of the alignment device inside a channel of the crimping device.

8. The alignment device of claim 7, wherein the at least one retaining member comprises a finger with a free end and attached end that are formed by a U-shaped slot in the cylindrical wall, wherein the free end includes a protrusion that protrudes radially outward from an outer surface of the free end, and wherein the free end is configured to deflect radially inward and outward relative to the attached end.

9. The alignment device of claim 1, wherein the support portion comprises one or more alignment indicators disposed at the narrower end, and wherein each alignment indicator is axially aligned with a corresponding alignment member of the one or more alignment members.

10. The alignment device of claim 1, wherein the coupling portion and the support portion are separate parts that are configured to be coupled to one another, and wherein the one or more alignment members are molded together with the coupling portion.

11. The alignment device of claim 10, wherein the coupling portion, including the one or more alignment members, comprises a first material, and wherein the support portion comprises a second material.

12. A system for crimping a prosthetic heart valve to a delivery apparatus, comprising:
the alignment device of claim 1; and
the crimping device which comprises a channel, wherein a first opening to the channel is disposed on a distal side of the crimping device, the first opening configured to receive the support portion of the alignment device therethrough, and wherein the crimping device includes a recess in the distal side that is configured to receive the coupling member of the alignment device therein.

13. A method comprising:
placing a prosthetic valve in a radially expanded configuration over a support surface of a tapered support portion of an alignment device and rotationally aligning the prosthetic valve on the support surface such that commissures of the prosthetic valve are axially aligned with alignment members of the alignment device, wherein the support portion extends from a proximal end of a coupling portion of the alignment device, wherein the alignment members extend axially outward from the proximal end of the coupling portion, over the support portion, and to the support surface, and wherein the coupling portion includes a coupling member that extends axially outward from a distal end of the coupling portion toward the support portion;
inserting the prosthetic valve placed over the support surface into a channel of a crimping device, the channel being surrounded by a plurality of pressing surfaces of the crimping device;
inserting the coupling member into a complementary recess in a distal face of the crimping device such that the alignment device is held in a specified circumferential orientation relative to the crimping device; and
radially compressing the prosthetic valve placed within the channel by actuating the crimping device to move the pressing surfaces radially inwardly,
wherein the radially compressing causes the alignment members to deflect radially inward and the alignment device to be moved axially out of and away from the prosthetic valve.

14. The method of claim 13, further comprising, prior to the radially compressing the prosthetic valve by actuating the crimping device, inserting a valve mounting portion of a delivery apparatus into the channel of the crimping device and through a central channel of the alignment device, and wherein the radially compressing the prosthetic valve includes radially compressing the prosthetic valve onto the valve mounting portion of the delivery apparatus.

15. The method of claim 13, wherein the coupling portion of the alignment device comprises a cylindrical wall and a retaining member disposed in the cylindrical wall, and further comprising sliding the retaining member along a surface defining the channel of the crimping device as the alignment device is inserted into the channel and moving the retaining member radially outward and into a slot in the crimping device that is radially offset from the channel such that the alignment device is retained in a specified axial orientation within the channel.

16. The method of claim 15, wherein the radially compressing further causes the alignment member to uncouple from the slot and allow the alignment device to be moved axially out of and away from the prosthetic valve.

17. An alignment device for a prosthetic heart valve, comprising:
a coupling portion comprising:
a cylindrical wall with a first end and a second end, wherein the second end comprises an axial wall, and wherein a plurality of indentations is disposed in a radially inward facing surface of the axial wall;
a coupling member extending axially from the first end toward the second end of the cylindrical wall, wherein the coupling member is configured to couple with a crimping device; and
one or more axially extending alignment members, each alignment member extending axially outward from the second end of the cylindrical wall;
a support portion that tapers from a wider, first end to a narrower, second end, wherein the support portion comprises:
an outward facing support surface that is configured to receive the prosthetic heart valve thereon, wherein the one or more axially extending alignment members extend over the support portion and to the support surface, each alignment member comprising a free end configured to abut an end of the prosthetic heart valve and be axially aligned with a commissure of the prosthetic heart valve when the prosthetic heart valve is arranged on the support surface; and
a plurality of tabs extending axially away from the first end of the support portion, wherein the plurality of tabs is configured to couple to the plurality of indentations such that the coupling portion and support portion are coupled to one another; and
a central channel extending through the coupling portion and the support portion, the central channel configured to receive a delivery apparatus for the prosthetic heart valve therethrough.

18. The alignment device of claim 17, wherein the coupling portion and the support portion are separately molded components and removable from one another.

19. The alignment device of claim 17, wherein the coupling portion comprises a first material, and wherein the support portion comprises a second material.

20. The alignment device of claim 19, wherein the second material has a higher durometer than the first material.
